(19) United States
(12) Patent Application Publication
(10) Pub. No.: US 2010/0291134 A1
(43) Pub. Date: Nov. 18, 2010

(54) TRUNCATED HEPATITIS C VIRUS NS5 DOMAIN AND FUSION PROTEINS COMPRISING SAME

(75) Inventors: Michael Houghton, Danville, CA (US); Angelica Medina-Selby, San Francisco, CA (US); Doris Coit, Petaluma, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: Chiron Corporation

(21) Appl. No.: 12/847,312

(22) Filed: Jul. 30, 2010

Related U.S. Application Data
(63) Continuation of application No. 11/131,901, filed on May 17, 2005, now abandoned.
(60) Provisional application No. 60/571,985, filed on May 17, 2004.

(51) Int. Cl. A61K 39/29; C07K 2/00; C07K 14/00; A61K 38/00; C07H 21/04; C12N 15/79; C12N 5/10; C12P 21/00; A61P 37/04; A61P 31/14

(52) U.S. Cl. 424/192.1; 530/300; 530/350; 514/1.1; 536/23.72; 435/320.1; 435/325; 435/69.1

(57) ABSTRACT

The invention provides truncated HCV NS5 polypeptides and fusion proteins comprising the truncated NS5 polypeptides, fused to at least one other HCV epitope derived from another region of the HCV polyprotein. The fusions can be used in methods of stimulating an immune response to HCV, for example a cellular immune response to HCV, such as activating hepatitis C virus (HCV)-specific T cells, including CD4⁺ and CD8⁺ T cells. The method can be used in model systems to develop HCV-specific immunogenic compositions, as well as to immunize a mammal against HCV.

### CROSS-REFERENCE TO RELATED APPLICATION

This application is a continuation of U.S. application Ser. No. 11/131,901, filed May 17, 2005, which application claims the benefit under 35 U.S.C. §119(e)(1) of provisional application 60/571,985 filed on May 17, 2004, which applications are incorporated herein by reference in their entireties.

### TECHNICAL FIELD

The present invention relates to hepatitis C virus (HCV) polypeptides. More particularly, the invention relates to truncated HCV NS5 polypeptides and fusion proteins comprising the truncated NS5 polypeptides. The proteins are useful for stimulating immune responses, such as cell-mediated immune responses, for priming and/or activating HCV-specific T cells, as well as for diagnostic reagents.

### BACKGROUND OF THE INVENTION

Hepatitis C virus (HCV) infection is an important health problem with approximately 1% of the world's population infected with the virus. Over 75% of acutely infected individuals eventually progress to a chronic carrier state that can result in cirrhosis, liver failure, and hepatocellular carcinoma. See, Alter et al. (1992) N. Engl. J. Med. 327:1899-1905; Resnick and Koff. (1993) Arch. Intern. Med. 153:1672-1677; Seeff (1995) Gastrointest. Dis. 6:20-27; Tong et al. (1995) N. Engl. J. Med. 332:1463-1466.

HCV was first identified and characterized as a cause of NANBH by Houghton et al. The viral genomic sequence of HCV is known, as are methods for obtaining the sequence. See, e.g., International Publication Nos. WO 89/04669; WO 90/11089; and WO 90/14436. HCV has a 9.5 kb positive-sense, single-stranded RNA genome and is a member of the Flaviridae family of viruses. At least six distinct, but related genotypes of HCV, based on phylogenetic analyses, have been identified (Simmonds et al., J. Gen. Virol. (1993) 74:2391-2399). The virus encodes a single polyprotein having more than 3000 amino acid residues (Choo et al., Science (1989) 244:359-362; Choo et al., Proc. Natl. Acad. Sci. USA (1991) 88:2451-2455; Han et al., Proc. Natl. Acad. Sci. USA (1991) 88:1711-1715). The polyprotein is processed co- and post-translationally into both structural and non-structural (NS) proteins.

In particular, as shown in FIG. 1, several proteins are encoded by the HCV genome. The order and nomenclature of the cleavage products of the HCV polyprotein is as follows: NH₂₋C-E1-E2-p7-NS2-NS3-NS4a-NS4b-NS5a-NS5b-COOH. Initial cleavage of the polyprotein is catalyzed by host proteases which liberate three structural proteins, the N-terminal nucleocapsid protein (termed “core”) and two envelope glycoproteins, “E1” (also known as E) and “E2” (also known as E2/NS1), as well as nonstructural (NS) proteins that contain the viral enzymes. The NS regions are termed NS2, NS3, NS4 and NS5. NS2 is an integral membrane protein with proteolytic activity and, in combination with NS3, cleaves the NS2-NS3 sissle bond which in turn generates the NS3 N-terminus and releases a large polyprotein that includes both serine protease and RNA helicase activities. The NS3 protease serves to process the remaining polyprotein. In these reactions, NS3 liberates an NS3 cofactor (NS4a), two proteins (NS4b and NS5a), and an RNA-dependent RNA polymerase (NS5b). Completion of polyprotein maturation is initiated by autocatalytic cleavage at the NS3-NS4a junction, catalyzed by the NS3 serine protease.

Despite extensive advances in the development of pharmaceuticals against certain viruses like HIV, control of acute and chronic HCV infection has had limited success (Hoofnagle and di Bisceglie (1997) N. Engl. J. Med. 336:347-356). In particular, generation of cellular immune responses, such as strong cytotoxic T lymphocyte (CTL) responses, is thought to be important for the control and eradication of HCV infections.

Immunogenic HCV fusion proteins capable of generating cellular immune responses are described in International Application WO/2004/005473 and U.S. Pat. Nos. 6,562,346; 6,514,731 and 6,428,792. Nevertheless, there remains a need in the art for additional effective methods of stimulating immune responses, such as cellular immune responses, to HCV.

### SUMMARY OF THE INVENTION

It is an object of the invention to provide reagents and methods for stimulating an immune response, such as a cellular immune response to HCV, such as priming and/or activating T cells which recognize epitopes of HCV polypeptides. It is also an object of the invention to provide compositions for the prevention and/or treatment of HCV infection. It is also an object of the invention to provide reagents and methods for use in diagnostic assays for detecting the presence of HCV in a biological sample.

Accordingly, in one embodiment, the invention is directed to a C-terminally truncated NS5 polypeptide, wherein the polypeptide comprises a full-length NS5a polypeptide and an N-terminal portion of an NS5b polypeptide. In certain embodiments, the polypeptide is truncated at a position between amino acid 2500 and the C-terminus, numbered relative to the full-length HCV-1 polyprotein, such as between amino acid 2900 and the C-terminus, or at the amino acid corresponding to the amino acid immediately following amino acid 2990, numbered relative to the full-length HCV-1 polyprotein.

In additional embodiments the polypeptide consists of an amino acid sequence corresponding to amino acids 1973-2990, numbered relative to the full-length HCV-1 polyprotein.

In further embodiments, the invention is directed to an immunogenic fusion protein comprising the C-terminally truncated NS5 polypeptide of any of the above embodiments, and at least one polypeptide derived from a region of the HCV polyprotein other than the NS5 region.

In yet additional embodiments, the protein further comprises a modified NS3 polypeptide comprising a substitution of an amino acid corresponding to His-1083, Asp-1105 and/or Ser-1165, numbered relative to the full-length HCV-1 polyprotein such that protease activity is inhibited when the modified NS3 polypeptide is present in an HCV fusion protein. In certain embodiments, the modified NS3 polypeptide comprises a substitution of an alanine for the amino acid corresponding to Ser-1165, numbered relative to the full-length HCV-1 polyprotein.

In further embodiments, the protein comprises a modified NS3 polypeptide, an NS4 polypeptide, and optionally an HCV core polypeptide.

In additional embodiments, the core polypeptide comprises a C-terminal truncation. In certain embodiments, the core polypeptide consists of the sequence of amino acids depicted at amino acid positions 1772-1892 of FIG. 3.

In yet further embodiments, the fusion protein further comprises an E2 polypeptide. In certain embodiments, the E2 polypeptide is a C-terminally truncated E2 polypeptide consisting of an amino acid sequence corresponding to amino acids 384-715, numbered relative to the full-length HCV-1 polyprotein.

In additional embodiments, the polypeptides present in the fusion are derived from the same HCV isolate. In other embodiments, at least one of the polypeptides present in the fusion is derived from a different isolate than the C-terminally truncated NS5 polypeptide.

In yet additional embodiments, the invention is directed to an immunogenic fusion protein consisting essentially of, in amino terminal to carboxy terminal direction:

(a) a modified NS3 polypeptide comprising a substitution of an alanine for the amino acid corresponding to Ser-1165, numbered relative to the full-length HCV-1 polyprotein such that protease activity is inhibited;

(b) an NS4 polypeptide;

(c) a C-terminally truncated NS5 polypeptide, wherein the NS5 polypeptide consists of an amino acid sequence corresponding to amino acids 1973-2990, numbered relative to the full-length HCV-1 polyprotein; and

(d) optionally, an HCV core polypeptide.

In yet further embodiments, the invention is directed to an immunogenic fusion protein consisting essentially of, in amino terminal to carboxy terminal direction:

(a) a C-terminally truncated E2 polypeptide consisting of an amino acid sequence corresponding to amino acids 384-715, numbered relative to the full-length HCV-1 polyprotein;

(b) a modified NS3 polypeptide comprising a substitution of an alanine for the amino acid corresponding to Ser-1165, numbered relative to the full-length HCV-1 polyprotein such that protease activity is inhibited;

(c) an NS4 polypeptide;

(d) a C-terminally truncated NS5 polypeptide, wherein the NS5 polypeptide consists of an amino acid sequence corresponding to amino acids 1973-2990, numbered relative to the full-length HCV-1 polyprotein; and

(e) optionally, an HCV core polypeptide.

In certain embodiments, the fusion proteins above comprise an HCV core polypeptide. In some embodiments, the core polypeptide comprises a C-terminal truncation, such a core polypeptide that consists of the sequence of amino acids depicted at amino acid positions 1772-1892 of FIG. 3.

In yet further embodiments, the invention is directed to a composition comprising a C-terminally truncated NS5 polypeptide according to any of the embodiments above, or a fusion protein according to any of the embodiments above, in combination with a pharmaceutically acceptable excipient. In certain embodiments, the compositions include an immunogenic HCV polypeptide, such as an HCV E1E2 complex. The E1E2 complex can be provided separately from the NS5 polypeptide or separately from the fusion protein including the NS5 polypeptide.

In additional embodiments, the invention is directed to a method of stimulating a cellular immune response in a vertebrate subject comprising administering to the subject a therapeutically effective amount of a composition as described above.

In further embodiments, the invention is directed to a method for producing a composition comprising combining a C-terminally truncated NS5 polypeptide according to any of the above embodiments, or a fusion protein according to any of the above embodiments, with a pharmaceutically acceptable excipient.

In yet additional embodiments, the invention is directed to a polynucleotide comprising a coding sequence encoding a C-terminally truncated NS5 polypeptide according to any of the above embodiments, or encoding an immunogenic fusion protein according to any of the above embodiments.

In further embodiments, the invention is directed to a recombinant vector comprising:

(a) a polynucleotide as described above; and

(b) at least one control element operably linked to the polynucleotide, whereby the coding sequence can be transcribed and translated in a host cell.

In additional embodiments, the invention is directed to a host cell comprising the recombinant vector described above.

In further embodiments, the invention is directed to a method for producing an immunogenic C-terminally truncated NS5 polypeptide or an immunogenic fusion protein comprising the polypeptide, the method comprising culturing a population of host cells as described above under conditions for producing the protein.

In additional embodiments, the invention is directed to a method for enhancing production of an HCV NS5 polypeptide comprising culturing a population of host cells as described above under conditions for producing the protein, wherein the protein is produced in greater amounts as compared to the amount of a full-length NS5 polypeptide produced under the same conditions.

These and other embodiments of the subject invention will readily occur to those of skill in the art in view of the disclosure herein.

### BRIEF DESCRIPTION OF THE FIGURES

FIG. 1 is a diagrammatic representation of the HCV genome, depicting the various regions of the HCV polyprotein.

FIG. 2 (SEQ ID NOS:3 and 4) depicts the DNA and corresponding amino acid sequence of a representative native, unmodified NS3 protease domain.

FIG. 3 (SEQ ID NOS:5 and 6) shows the DNA and corresponding amino acid sequence of a representative modified fusion protein, with the NS3 protease domain deleted from the N-terminus and including amino acids 1-121 of Core on the C-terminus.

FIGS. 4A and 4B show a comparison of expression levels of NS5tCore121 (amino acids 1973-2990 of NS5 and 1-121 of core) and NS5Core121 (full-length NS5, amino acids 1973-3011 of NS5 and 1-121 of core) in S. cerevisiae strain AD3. FIG. 4A shows expression levels at 25° C. and FIG. 4B shows expression levels at 30° C. Lane 1, standard; Lane 2, plasmid control; Lane 3, plasmid encoding NS5tCore121 (clone 6); Lane 4, plasmid encoding NS5tCore121 (clone 7); Lane 5, plasmid encoding NS5Core121 (clone 8); Lane 6, plasmid encoding NS5Core121 (clone 9); Lane 7, standard.

FIGS. 5A-5E (SEQ ID NOS:7 and 8) show the DNA and corresponding amino acid sequence of a representative fusion protein that includes a C-terminally truncated NS5 polypeptide with the C-terminus of the NS5 polypeptide fused to a core polypeptide. In particular, the C-terminally truncated NS5 polypeptide includes amino acids 1973-2990 of the HCV polyprotein, numbered relative to HCV-1 (see, Choo et al. (1991) Proc. Natl. Acad. Sci. USA 88:2451-2455), fused to a core polypeptide that includes amino acids 1-121 of the HCV polyprotein.

### DETAILED DESCRIPTION OF THE INVENTION

The practice of the present invention will employ, unless otherwise indicated, conventional methods of chemistry, biochemistry, recombinant DNA techniques and immunology, within the skill of the art. Such techniques are explained fully in the literature. See, e.g., Sambrook, et al., Molecular Cloning: A Laboratory Manual (2nd Edition); Methods In Enzymology (S. Colowick and N. Kaplan eds., Academic Press, Inc.); DNA Cloning, Vols. I and II (D. N. Glover ed.); Oligonucleotide Synthesis (M. J. Gait ed.); Nucleic Acid Hybridization (B. D. Hames & S. J. Higgins eds.); Animal Cell Culture (R. K. Freshney ed.); Perbal, B., A Practical Guide to Molecular Cloning.

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “a polypeptide” includes a mixture of two or more polypeptides, and the like.

The following amino acid abbreviations are used throughout the text:
Alanine: Ala (A) Arginine: Arg (R)
Asparagine: Asn (N) Aspartic acid: Asp (D)
Cysteine: Cys (C) Glutamine: Gln (Q)
Glutamic acid: Glu (E) Glycine: Gly (G)
Histidine: H is (H) Isoleucine: Ile (I)
Leucine: Leu (L) Lysine: Lys (K)
Methionine: Met (M) Phenylalanine: Phe (F)
Proline: Pro (P) Serine: Ser (S)
Threonine: Thr (T) Tryptophan: Trp (W)
Tyrosine: Tyr (Y) Valine: Val (V)

### I. DEFINITIONS

In describing the present invention, the following terms will be employed, and are intended to be defined as indicated below.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification.

An HCV polypeptide is a polypeptide, as defined above, derived from the HCV polyprotein. The polypeptide need not be physically derived from HCV, but may be synthetically or recombinantly produced. Moreover, the polypeptide may be derived from any of the various HCV strains and isolates including isolates having any of the 6 genotypes of HCV described in Simmonds et al., J. Gen. Virol. (1993) 74:2391-2399 (e.g., strains 1, 2, 3, 4 etc.), as well as newly identified isolates, and subtypes of these isolates, such as HCV1a, HCV 1b etc. A number of conserved and variable regions are known between these strains and, in general, the amino acid sequences of epitopes derived from these regions will have a high degree of sequence homology, e.g., amino acid sequence homology of more than 30%, preferably more than 40%, when the two sequences are aligned. Thus, for example, the term “NS5” polypeptide refers to native NS5 from any of the various HCV strains, as well as NS5 analogs, muteins and immunogenic fragments, as defined further below.

The terms “analog” and “mutein” refer to biologically active derivatives of the reference molecule, or fragments of such derivatives, that retain desired activity, such as the ability to stimulate a cell-mediated immune response, as defined below. In the case of a modified NS3, an “analog” or “mutein” refers to an NS3 molecule that lacks its native proteolytic activity. In general, the term “analog” refers to compounds having a native polypeptide sequence and structure with one or more amino acid additions, substitutions (generally conservative in nature, or in the case of modified NS3, non-conservative in nature at the active proteolytic site) and/or deletions, relative to the native molecule, so long as the modifications do not destroy immunogenic activity. The term “mutein” refers to peptides having one or more peptide mimics (“peptoids”). Preferably, the analog or mutein has at least the same immunoactivity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “C-terminally truncated NS5 polypeptide” is meant an NS5 polypeptide that comprises a full-length NS5a polypeptide and an N-terminal portion of an NS5b polypeptide, but not the entire NS5b region. Particular examples of C-terminally truncated NS5 polypeptides are provided below.

By “modified NS3” is meant an NS3 polypeptide with a modification such that protease activity of the NS3 polypeptide is disrupted. The modification can include one or more amino acid additions, substitutions (generally non-conservative in nature) and/or deletions, relative to the native molecule, wherein the protease activity of the NS3 polypeptide is disrupted. Methods of measuring protease activity are discussed further below.

By “fragment” is intended a polypeptide consisting of only a part of the intact full-length polypeptide sequence and structure. The fragment can include a C-terminal deletion and/or an N-terminal deletion of the native polypeptide. An “immunogenic fragment” of a particular HCV protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains immunogenic activity, as measured by the assays described herein.

The term “epitope” as used herein refers to a sequence of at least about 3 to 5, preferably about 5 to 10 or 15, and not more than about 1,000 amino acids (or any integer therebetween), which define a sequence that by itself or as part of a larger sequence, binds to an antibody generated in response to such sequence. There is no critical upper limit to the length of the fragment, which may comprise nearly the full-length of the protein sequence, or even a fusion protein comprising two or more epitopes from the HCV polyprotein. An epitope for use in the subject invention is not limited to a polypeptide having the exact sequence of the portion of the parent protein from which it is derived. Indeed, viral genomes are in a state of constant flux and contain several variable domains which exhibit relatively high degrees of variability between isolates. Thus the term “epitope” encompasses sequences identical to the native sequence, as well as modifications to the native sequence, such as deletions, additions and substitutions (generally conservative in nature).

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci. USA (1981) 78:3824-3828 for determining antigenicity profiles, and the Kyte-Doolittle technique, Kyte et al., J. Mol. Biol. (1982) 157:105-132 for hydropathy plots.

For a description of various HCV epitopes, see, e.g., Chien et al., Proc. Natl. Acad. Sci. USA (1992) 89:10011-10015; Chien et al., J. Gastroent. Hepatol. (1993) 8:S33-39; Chien et al., International Publication No. WO 93/00365; Chien, D. Y., International Publication No. WO 94/01778; and U.S. Pat. Nos. 6,280,927 and 6,150,087, incorporated herein by reference in their entireties.

As used herein the term “T-cell epitope” refers to a feature of a peptide structure which is capable of inducing T-cell immunity towards the peptide structure or an associated hapten. T-cell epitopes generally comprise linear peptide determinants that assume extended conformations within the peptide-binding cleft of MHC molecules, (Unanue et al., Science (1987) 236:551-557). Conversion of polypeptides to MHC class II-associated linear peptide determinants (generally between 5-14 amino acids in length) is termed “antigen processing” which is carried out by antigen presenting cells (APCs). More particularly, a T-cell epitope is defined by local features of a short peptide structure, such as primary amino acid sequence properties involving charge and hydrophobicity, and certain types of secondary structure, such as helicity, that do not depend on the folding of the entire polypeptide. Further, it is believed that short peptides capable of recognition by helper T-cells are generally amphipathic structures comprising a hydrophobic side (for interaction with the MHC molecule) and a hydrophilic side (for interacting with the T-cell receptor), (Margalit et al., Computer Prediction of T-cell Epitopes, New Generation Vaccines Marcel-Dekker, Inc, ed. G. C. Woodrow et al., (1990) pp. 109-116) and further that the amphipathic structures have an α-helical configuration (see, e.g., Spouge et al., J. Immunol. (1987) 138:204-212; Berkower et al., J. Immunol. (1986) 136:2498-2503).

Hence, segments of proteins that include T-cell epitopes can be readily predicted using numerous computer programs. (See e.g., Margalit et al., Computer Prediction of T-cell Epitopes, New Generation Vaccines Marcel-Dekker, Inc, ed. G. C. Woodrow et al., (1990) pp. 109-116). Such programs generally compare the amino acid sequence of a peptide to sequences known to induce a T-cell response, and search for patterns of amino acids which are believed to be required for a T-cell epitope.

An “immunological response” to an HCV antigen (including both polypeptide and polynucleotides encoding polypeptides that are expressed in vivo) or composition is the development in a subject of a humoral and/or a cellular immune response to molecules present in the composition of interest. For purposes of the present invention, a “Immoral immune response” refers to an immune response mediated by antibody molecules, while a “cellular immune response” is one mediated by T lymphocytes and/or other white blood cells. One important aspect of cellular immunity involves an antigen-specific response by cytolytic T cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Both CD8+ and CD4+T cells are capable of killing HCV-infected cells. Another aspect of cellular immunity involves an antigen-specific response by helper T cells. Helper T cells act to help stimulate the function, and focus the activity of, nonspecific effector cells against cells displaying peptide antigens in association with MHC molecules on their surface. A “cellular immune response” also refers to the production of antiviral cytokines, chemokines and other such molecules produced by activated T cells and/or other white blood cells, including those derived from CD4+ and CD8+T cells, including, but not limited to IFN-γ and TNF-α.

A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T lymphocytes can be generated to allow for the future protection of an immunized host.

The ability of a particular antigen to stimulate a cell-mediated immunological response may be determined by a number of assays, such as by lymphoproliferation (lymphocyte activation) assays, CTL cytotoxic cell assays, or by assaying for T lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376; and the examples below.

Thus, an immunological response as used herein may be one which stimulates the production of CTLs, and/or the production or activation of helper T cells. The antigen of interest may also elicit an antibody-mediated immune response. Hence, an immunological response may include one or more of the following effects: the production of antibodies by B-cells; and/or the activation of suppressor T cells and/or γδ T cells directed specifically to an antigen or antigens present in the composition or vaccine of interest. These responses may serve to neutralize infectivity, and/or mediate antibody-complement, or antibody dependent cell cytotoxicity (ADCC) to provide protection (i.e., prophylactic) or alleviation of symptoms (i.e., therapeutic) to an immunized host. Such responses can be determined using standard immunoassays and neutralization assays, well known in the art.

By “equivalent antigenic determinant” is meant an antigenic determinant from different sub-species or strains of HCV, such as from strains 1, 2, 3, etc., of HCV which antigenic determinants are not necessarily identical due to sequence variation, but which occur in equivalent positions in the HCV sequence in question. In general the amino acid sequences of equivalent antigenic determinants will have a high degree of sequence homology, e.g., amino acid sequence homology of more than 30%, usually more than 40%, such as more than 60%, and even more than 80-90% homology, when the two sequences are aligned.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

A “nucleic acid” molecule or “polynucleotide” can include both double- and single-stranded sequences and refers to, but is not limited to, cDNA from viral, procaryotic or eucaryotic mRNA, genomic DNA sequences from viral (e.g. DNA viruses and retroviruses) or procaryotic DNA, and especially synthetic DNA sequences. The term also captures sequences that include any of the known base analogs of DNA and RNA.

“Operably linked” refers to an arrangement of elements wherein the components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can transcribed introns, and the promoter sequence can still be considered “operably linked” to the coding sequence.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

A “control element” refers to a polynucleotide sequence which aids in the expression of a coding sequence to which it is linked. The term includes promoters, transcription termination sequences, upstream regulatory domains, polyadenylation signals, untranslated regions, including 5′-UTRs and 3′-UTRs and when appropriate, leader sequences and enhancers, which collectively provide for the transcription and translation of a coding sequence in a host cell.

A “promoter” as used herein is a DNA regulatory region capable of binding RNA polymerase in a host cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

A control sequence “directs the transcription” of a coding sequence in a cell when RNA polymerase will bind the promoter sequence and transcribe the coding sequence into mRNA, which is then translated into the polypeptide encoded by the coding sequence.

“Expression cassette” or “expression construct” refers to an assembly which is capable of directing the expression of the sequence(s) or gene(s) of interest. The expression cassette includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

“Transformation,” as used herein, refers to the insertion of an exogenous polynucleotide into a host cell, irrespective of the method used for insertion: for example, transformation by direct uptake, transfection, infection, and the like. For particular methods of transfection, see further below. The exogenous polynucleotide may be maintained as a nonintegrated vector, for example, an episome, or alternatively, may be integrated into the host genome.

A “host cell” is a cell which has been transformed, or is capable of transformation, by an exogenous DNA sequence.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “purified” as used herein preferably means at least 75% by weight, more preferably at least 85% by weight, more preferably still at least 95% by weight, and most preferably at least 98% by weight, of biological macromolecules of the same type are present.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98%, or more, sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be readily found at the NCBI internet site.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

By “nucleic acid immunization” is meant the introduction of a nucleic acid molecule encoding one or more selected immunogens into a host cell, for the in vivo expression of the immunogen or immunogens. The nucleic acid molecule can be introduced directly into the recipient subject, such as by injection, inhalation, oral, intranasal and mucosal administration, or the like, or can be introduced ex vivo, into cells which have been removed from the host. In the latter case, the transformed cells are reintroduced into the subject where an immune response can be mounted against the antigen encoded by the nucleic acid molecule.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

By “vertebrate subject” is meant any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species, since the immune systems of all of these vertebrates operate similarly.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

The present invention pertains to HCV NS5 polypeptides that comprise a full-length HCV NS5a polypeptide and a portion of an HCV NS5b polypeptide with a C-terminal truncation. The invention also relates to fusion proteins and polynucleotides encoding the same, comprising the truncated NS5 polypeptide and at least one other HCV polypeptide from the HCV polyprotein. The proteins of the present invention can be used to stimulate immunological responses, such as a humoral and/or cellular immune response, for example to activate HCV-specific T cells, i.e., T cells which recognize epitopes of these polypeptides and/or to elicit the production of helper T cells and/or to stimulate the production of antiviral cytokines, chemokines, and the like. Activation of HCV-specific T cells by such fusion proteins provides both in vitro and in vivo model systems for the development of HCV vaccines, particularly for identifying HCV polypeptide epitopes associated with a response. The proteins can also be used to generate an immune response against HCV in a mammal, for example a CTL response, and/or to prime CD8+ and CD4+T cells to produce antiviral agents, for either therapeutic or prophylactic purposes.

The proteins are therefore useful for treating and/or preventing HCV infection. The proteins can be used alone or in combination with one or more bacterial or viral immunogens. The combinations may include multiple immunogens from the same pathogen, multiple immunogens from different pathogens or multiple immunogens from the same and from different pathogens. Thus, bacterial, viral, and/or other immunogens may be included in the same composition as the NS5 polypeptides, or may be administered to the same subject separately, or may even be included in fusion proteins with the NS5 polypeptides. As described further below, particularly useful are combinations of the NS5 polypeptides with other HCV immunogens.

Moreover, the proteins of the present invention can also be used as diagnostic reagents to detect HCV infection in a biological sample.

In order to further an understanding of the invention, a more detailed discussion is provided below regarding fusion proteins for use in the subject compositions, as well as production of the proteins, compositions comprising the same and methods of using the proteins.

### Fusion Proteins

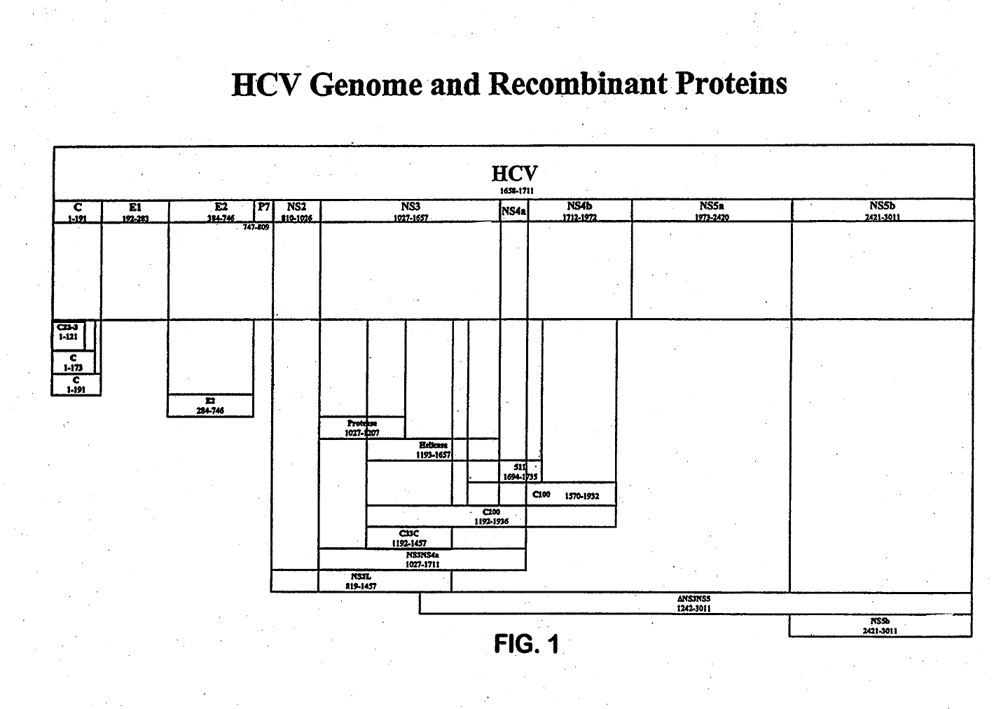

The genomes of HCV strains contain a single open reading frame of approximately 9,000 to 12,000 nucleotides, which is transcribed into a polyprotein. As shown in FIG. 1 and Table 1, an HCV polyprotein, upon cleavage, produces at least ten distinct products, in the order of NH₂₋Core-E1-E2-p7-NS2-NS3-NS4a-NS4b-NS5a-NS5b-COOH. The core polypeptide occurs at positions 1-191, numbered relative to HCV-1 (see, Choo et al. (1991) Proc. Natl. Acad. Sci. USA 88:2451-2455, for the HCV-1 genome). This polypeptide is further processed to produce an HCV polypeptide with approximately amino acids 1-173. The envelope polypeptides, E1 and E2, occur at about positions 192-383 and 384-746, respectively. The P7 domain is found at about positions 747-809. NS2 is an integral membrane protein with proteolytic activity and is found at about positions 810-1026 of the polyprotein. NS2, in combination with NS3, (found at about positions 1027-1657), cleaves the NS2-NS3 sissle bond which in turn generates the NS3 N-terminus and releases a large polyprotein that includes both serine protease and RNA helicase activities. The NS3 protease, found at about positions 1027-1207, serves to process the remaining polyprotein. The helicase activity is found at about positions 1193-1657. NS3 liberates an NS3 cofactor (NS4a, found about positions 1658-1711), two proteins (NS4b found at about positions 1712-1972, and NS5a found at about positions 1973-2420), and an RNA-dependent RNA polymerase (NS5b found at about positions 2421-3011). Completion of polyprotein maturation is initiated by autocatalytic cleavage at the NS3-NS4a junction, catalyzed by the NS3 serine protease.

TABLE 1

| Domain | Approximate Boundaries* |
|---|---|
| C (core) | 1-191 |
| E1 | 192-383 |
| E2 | 384-746 |
| P7 | 747-809 |
| NS2 | 810-1026 |
| NS3 | 1027-1657 |
| NS4a | 1658-1711 |
| NS4b | 1712-1972 |
| NS5a | 1973-2420 |
| NS5b | 2421-3011 |

*Numbered relative to HCV-1. See, Choo et al. (1991) Proc. Natl. Acad. Sci. USA 88: 2451-2455.

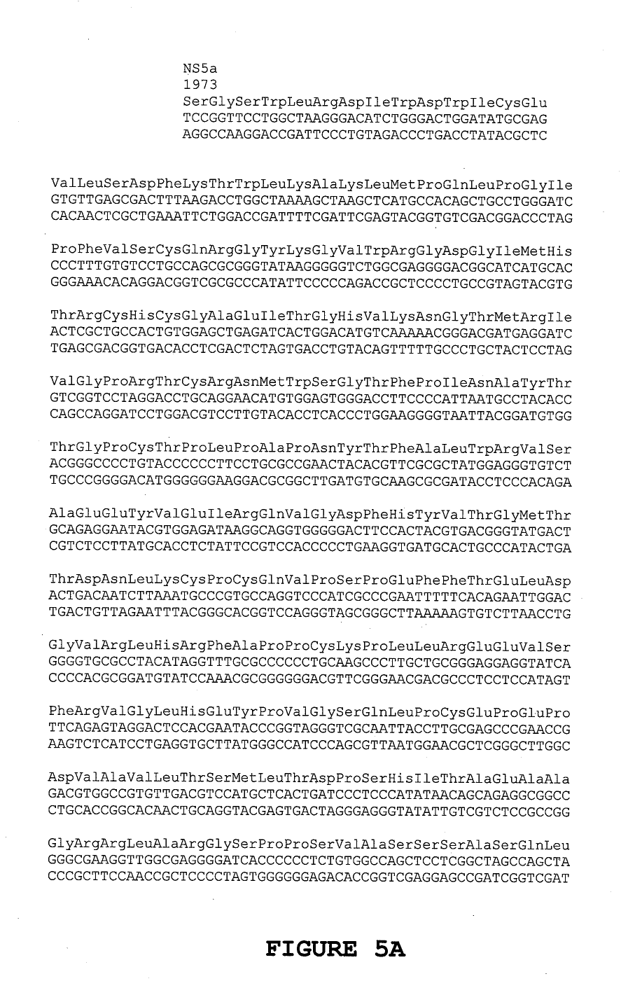

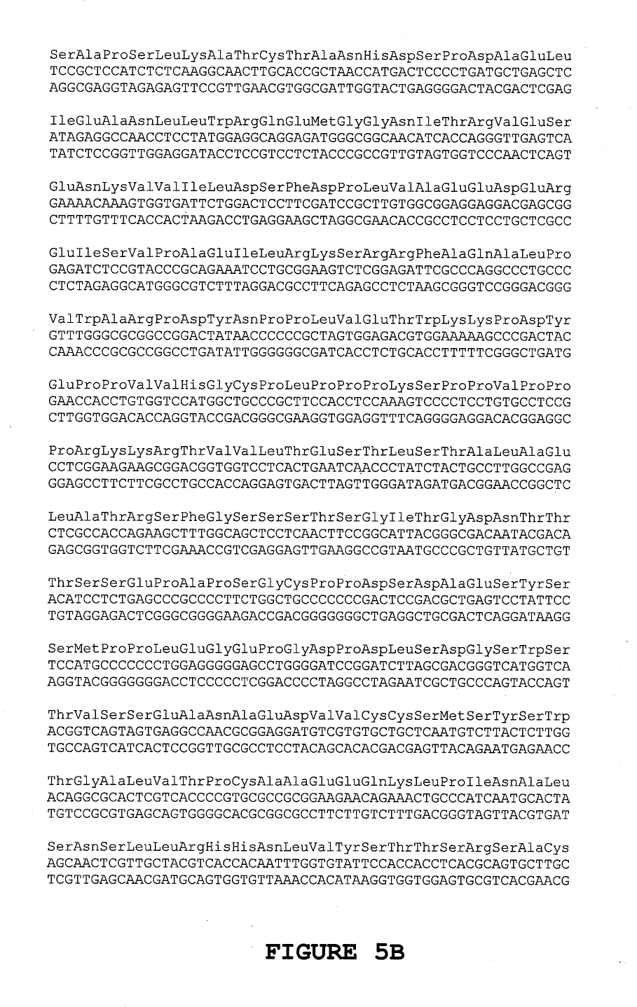

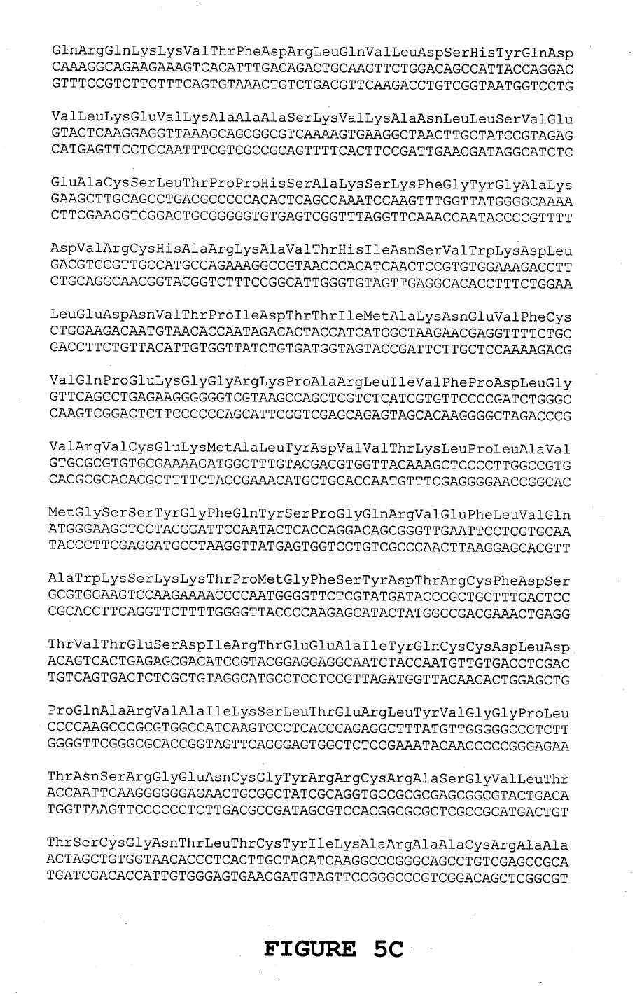

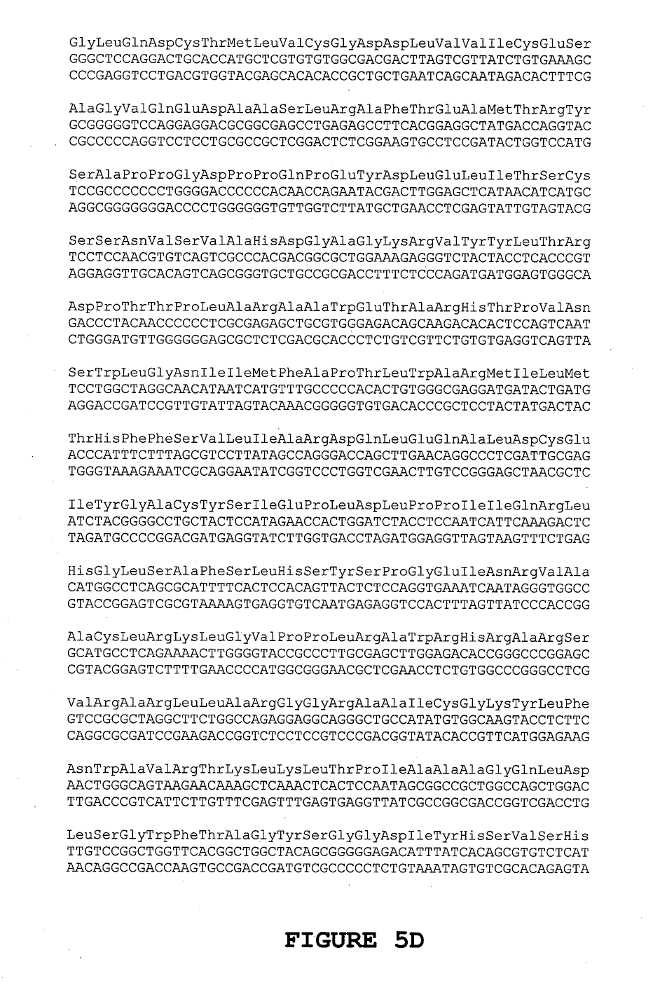

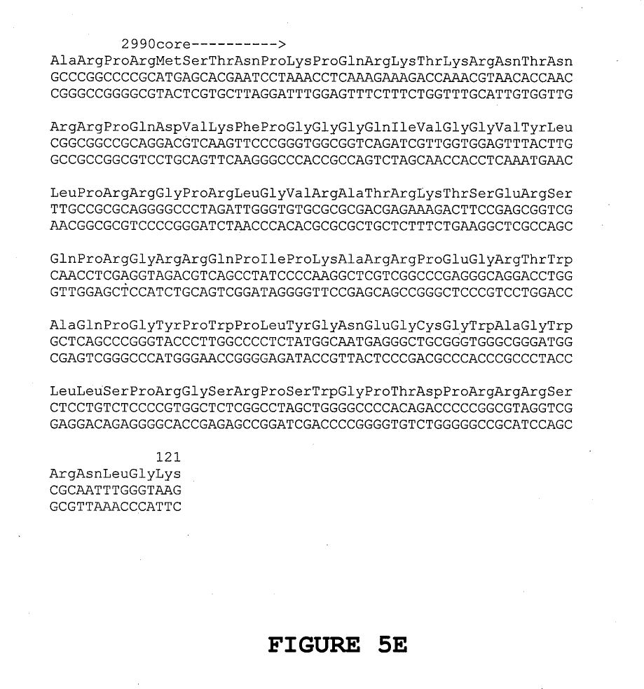

Fusion proteins of the invention include a C-terminally truncated NS5 polypeptide (also referred to herein as “NS5t”). In particular, the C-terminally truncated NS5 polypeptide comprises a full-length NS5a polypeptide and an N-terminal portion of an NS5b polypeptide. The C-terminally truncated polypeptide can be truncated at any position between amino acid 2500 and the C-terminus, numbered relative to the full-length HCV-1 polyprotein, such as after amino acid 2505 . . . 2550 . . . 2600 . . . 2650 . . . 2700 . . . 2750 . . . 2800 . . . 2850 . . . 2900 . . . 2950 . . . 2960 . . . 2970 . . . 2975 . . . 2980 . . . 2985 . . . 2990 . . . 2995 . . . 3000, etc, numbered relative to the full-length HCV-1 sequence. It is readily apparent that the molecule can be truncated at any amino acid between 2500 and 3010, numbered relative to the full-length HCV-1 sequence. One particularly preferred NS5 polypeptide is truncated at the amino acid corresponding to the amino acid immediately following amino acid 2990, numbered relative to the full-length HCV-1 polyprotein, and comprises an amino acid sequence corresponding to amino acids 1973-2990, numbered relative to the full-length HCV-1 polyprotein. The sequence for such a construct is shown at amino acid positions 1-1018 of SEQ ID NO:8 (labeled as amino acids 1973-2990 in FIGS. 5A-5E). The fusions of the invention optionally have an N-terminal methionine for expression.

The C-terminally truncated NS5 polypeptides can be used alone, in compositions described below, or in combination with one or more other HCV immunogenic polypeptides derived from any of the various domains of the HCV polyprotein. The additional HCV polypeptides can be provided separately or in the fusion. In fact, the fusion can include all the regions of the HCV polyprotein. These polypeptides may be derived from the same HCV isolate as the NS5 polypeptide, or from different strains and isolates including isolates having any of the various HCV genotypes, to provide increased protection against a broad range of HCV genotypes. Additionally, polypeptides can be selected based on the particular viral clades endemic in specific geographic regions where vaccine compositions containing the fusions will be used. It is readily apparent that the subject fusions provide an effective means of treating HCV infection in a wide variety of contexts.

Thus, NS5t can be included in a fusion protein comprising any combination of NS5t with one or more immunogenic HCV proteins from other domains in the HCV polyprotein, i.e., an NS5t combined with an E1, E2, p7, NS2, NS3, NS4, and/or a core polypeptide. These regions need not be in the order in which they occur naturally. Moreover, each of these regions can be derived from the same or a different HCV isolate. The various HCV polypeptides present in the various fusions described herein can either be full-length polypeptides or portions thereof. The portions of the HCV polypeptides making up the fusion protein generally comprise at least one epitope, which is recognized by a T cell receptor on an activated T cell, such as 2152-HEYPVGSQL-2160 (SEQ ID NO:1) and/or 2224-AELIEANLLWRQEMG-2238 (SEQ ID NO:2). Epitopes can be identified by several methods. For example, the individual polypeptides or fusion proteins comprising any combination of the above, can be isolated, by, e.g., immunoaffinity purification using a monoclonal antibody for the polypeptide or protein. The isolated protein sequence can then be screened by preparing a series of short peptides by proteolytic cleavage of the purified protein, which together span the entire protein sequence. By starting with, for example, 100-mer polypeptides, each polypeptide can be tested for the presence of epitopes recognized by a T-cell receptor on an HCV-activated T cell, progressively smaller and overlapping fragments can then be tested from an identified 100-mer to map the epitope of interest.

Epitopes recognized by a T-cell receptor on an HCV-activated T cell can be identified by, for example, a ⁵¹Cr release assay or by a lymphoproliferation assay (see the examples). In a ⁵¹Cr release assay, target cells can be constructed that display the epitope of interest by cloning a polynucleotide encoding the epitope into an expression vector and transforming the expression vector into the target cells. HCV-specific CD8⁺T cells will lyse target cells displaying, for example, one or more epitopes from one or more regions of the HCV polyprotein found in the fusion, and will not lyse cells that do not display such an epitope. In a lymphoproliferation assay, HCV-activated CD4⁺T cells will proliferate when cultured with, for example, one or more epitopes from one or more regions of the HCV polyprotein found in the fusion, but not in the absence of an HCV epitopic peptide.

The various HCV polypeptides can occur in any order in the fusion protein. If desired, at least 2, 3, 4, 5, 6, 7, 8, 9, or 10 or more of one or more of the polypeptides may occur in the fusion protein. Multiple viral strains of HCV occur, and HCV polypeptides of any of these strains can be used in a fusion protein.

Nucleic acid and amino acid sequences of a number of HCV strains and isolates, including nucleic acid and amino acid sequences of the various regions of the HCV polyprotein, including Core, NS2, p7, E1, E2, NS3, NS4, NS5a, NS5b genes and polypeptides have been determined. For example, isolate HCV J1.1 is described in Kubo et al. (1989) Japan. Nucl. Acids Res. 17:10367-10372; Takeuchi et al. (1990) Gene 91:287-291; Takeuchi et al. (1990) J. Gen. Virol. 71:3027-3033; and Takeuchi et al. (1990) Nucl. Acids Res. 18:4626. The complete coding sequences of two independent isolates, HCV-J and BK, are described by Kato et al., (1990) Proc. Natl. Acad. Sci. USA 87:9524-9528 and Takamizawa et al., (1991) J. Virol. 65:1105-1113 respectively.

Publications that describe HCV-1 isolates include Choo et al. (1990) Brit. Med. Bull. 46:423-441; Choo et al. (1991) Proc. Natl. Acad. Sci. USA 88:2451-2455 and Han et al. (1991) Proc. Natl. Acad. Sci. USA 88:1711-1715. HCV isolates HC-J1 and HC-J4 are described in Okamoto et al. (1991) Japan J. Exp. Med. 60:167-1′77. HCV isolates HCT 18˜, HCT 23, Th, HCT 27, EC 1 and EC 10 are described in Weiner et al. (1991) Virol. 180:842-848. HCV isolates Pt-1, HCV-K1 and HCV-K2 are described in Enomoto et al. (1990) Biochem. Biophys. Res. Commun. 170:1021-1025. HCV isolates A, C, D & E are described in Tsukiyama-Kohara et al. (1991) Virus Genes 5:243-254.

As explained above, each of the components of a fusion protein can be obtained from the same HCV strain or isolate or from different HCV strains or isolates. For example, the NS5 polypeptide can be derived from a first strain of HCV, and the other HCV polypeptides present can be derived from a second strain of HCV. Alternatively, one or more of the other HCV polypeptides, for example NS2, NS3, NS4, Core, p′7, E1 and/or E2, if present, can be derived from a first strain of HCV, and the remaining HCV polypeptides can be derived from a second strain of HCV. Additionally, each or the HCV polypeptides present can be derived from different HCV strains.

For a description of various HCV epitopes from the HCV regions for use in the subject fusions, see, e.g., Chien et al., Proc. Natl. Acad. Sci. USA (1992) 89:10011-10015; Chien et al., J. Gastroent. Hepatol. (1993) 8:S33-39; Chien et al., International Publication No. WO 93/00365; Chien, D. Y., International Publication No. WO 94/01778; and U.S. Pat. Nos. 6,280,927 and 6,150,087, incorporated herein by reference in their entireties.

For example, fusions can comprise the C-terminally truncated NS5 polypeptide and an NS3 polypeptide. The NS3 polypeptide can be modified to inhibit protease activity, such that further cleavage of the fusion is inhibited (also referred to herein as “NS3*”). The NS3 polypeptide can be modified by deletion of all or a portion of the NS3 protease domain. Alternatively, proteolytic activity can be inhibited by substitutions of amino acids within active regions of the protease domain. Finally, additions of amino acids to active regions of the domain, such that the catalytic site is modified, will also serve to inhibit proteolytic activity.

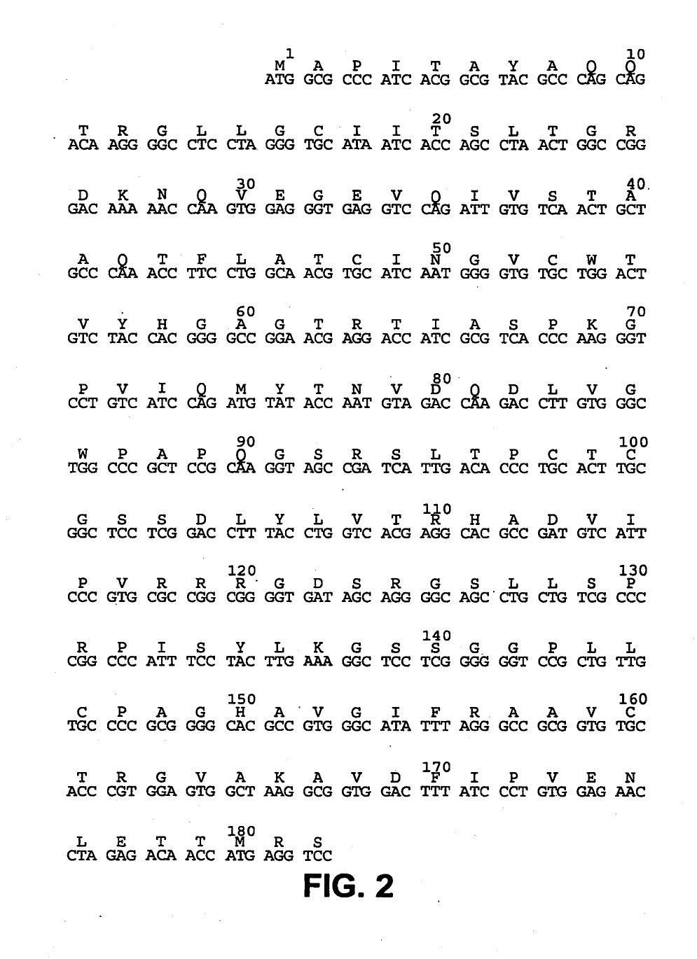

As explained above, the protease activity is found at about amino acid positions 1027-1207, numbered relative to the full-length HCV-1 polyprotein (see, Choo et al., Proc. Natl. Acad. Sci. USA (1991) 88:2451-2455), positions 2-182 of FIG. 2. The structure of the NS3 protease and active site are known. See, e.g., De Francesco et al., Antivir. Ther. (1998) 3:99-109; Koch et al., Biochemistry (2001) 40:631-640. Thus, deletions or modifications to the native sequence will typically occur at or near the active site of the molecule. Particularly, it is desirable to modify or make deletions to one or more amino acids occurring at positions 1- or 2-182, preferably 1- or 2-170, or 1- or 2-155 of FIG. 2. Preferred modifications are to the catalytic triad at the active site of the protease, i.e., H, D and/or S residues, in order to inactivate the protease. These residues occur at positions 1083, 1105 and 1165, respectively, numbered relative to the full-length HCV polyprotein (positions 58, 80 and 140, respectively, of FIG. 2). Such modifications will suppress proteolytic cleavage while maintaining T-cell epitopes. One particularly preferred modification is a substitution of Ser-1165 with Ala. One of skill in the art can readily determine portions of the NS3 protease to delete in order to disrupt activity. The presence or absence of activity can be determined using methods known to those of skill in the art.

For example, protease activity or lack thereof may be determined using the procedure described below in the examples, as well as using assays well known in the art. See, e.g., Takeshita et al., Anal. Biochem. (1997) 247:242-246; Kakiuchi et al., J. Biochem. (1997) 122:749-755; SalI et al., Biochemistry (1998) 37:3392-3401; Cho et al., J. Virol. Meth. (1998) 72:109-115; Cerretani et al., Anal. Biochem. (1999) 266:192-197; Zhang et al., Anal. Biochem. (1999) 270:268-275; Kakiuchi et al., J. Virol. Meth. (1999) 80:77-84; Fowler et al., J. Biomol. Screen. (2000) 5:153-158; and Kim et al., And Biochem. (2000) 284:42-48.

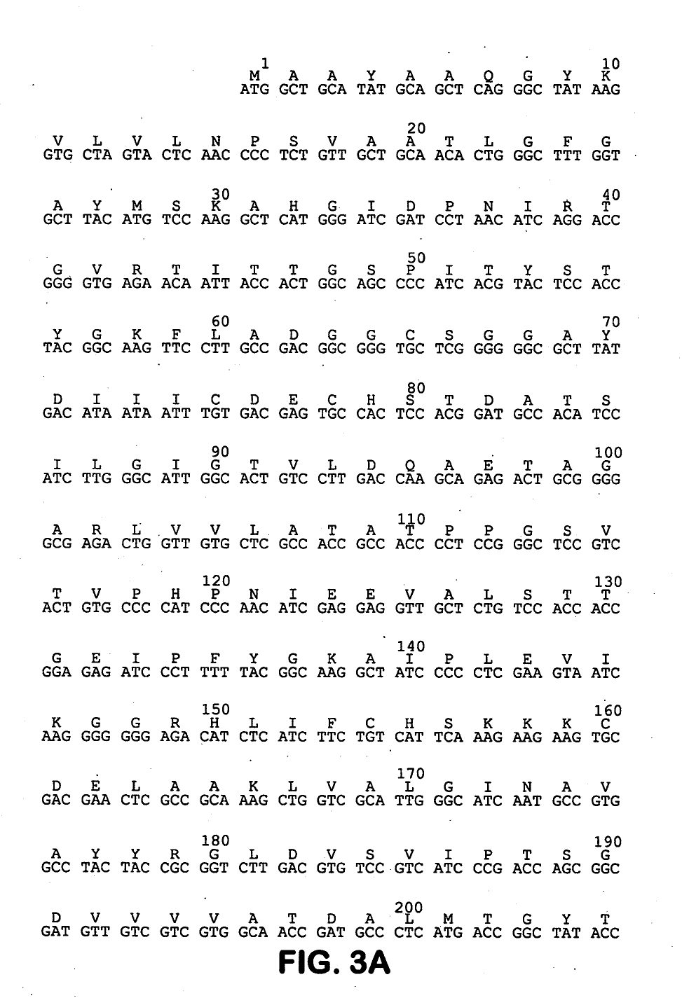

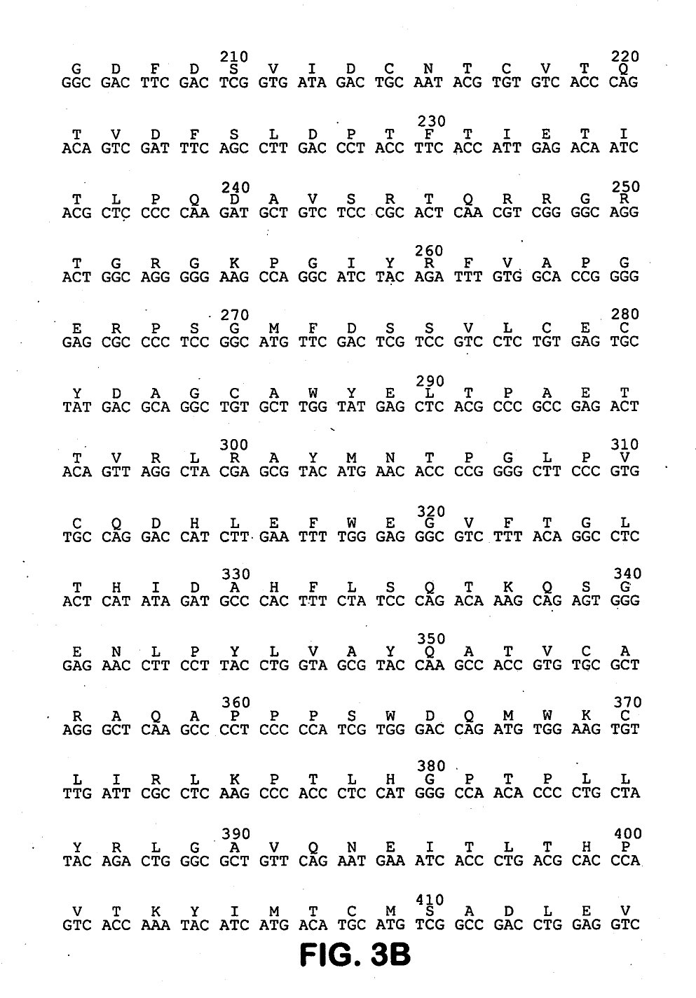

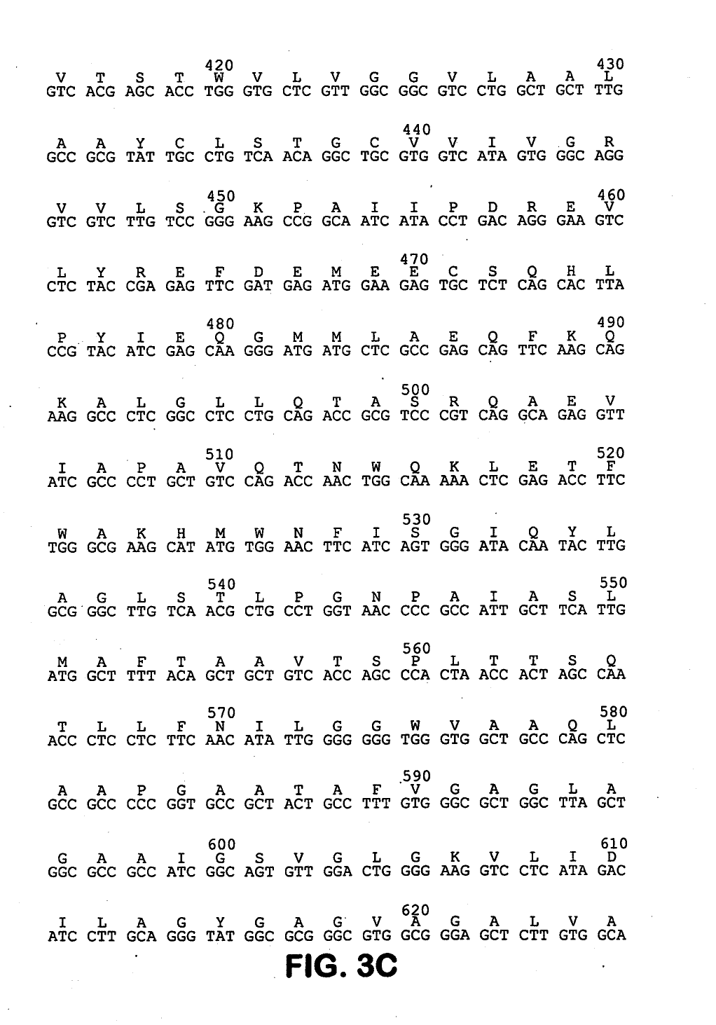

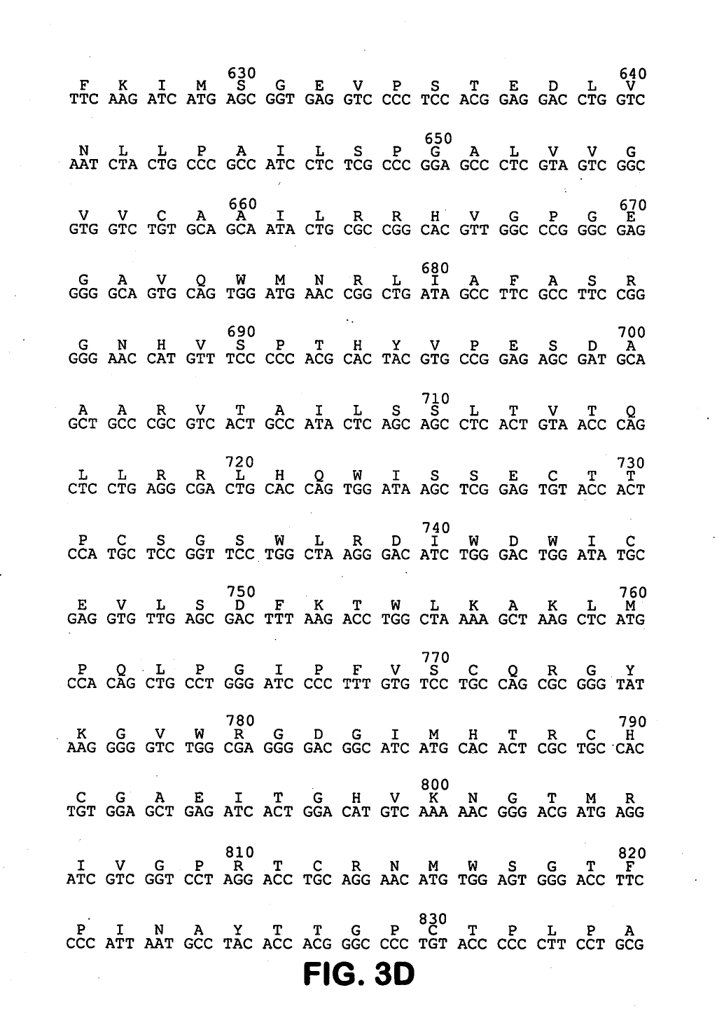

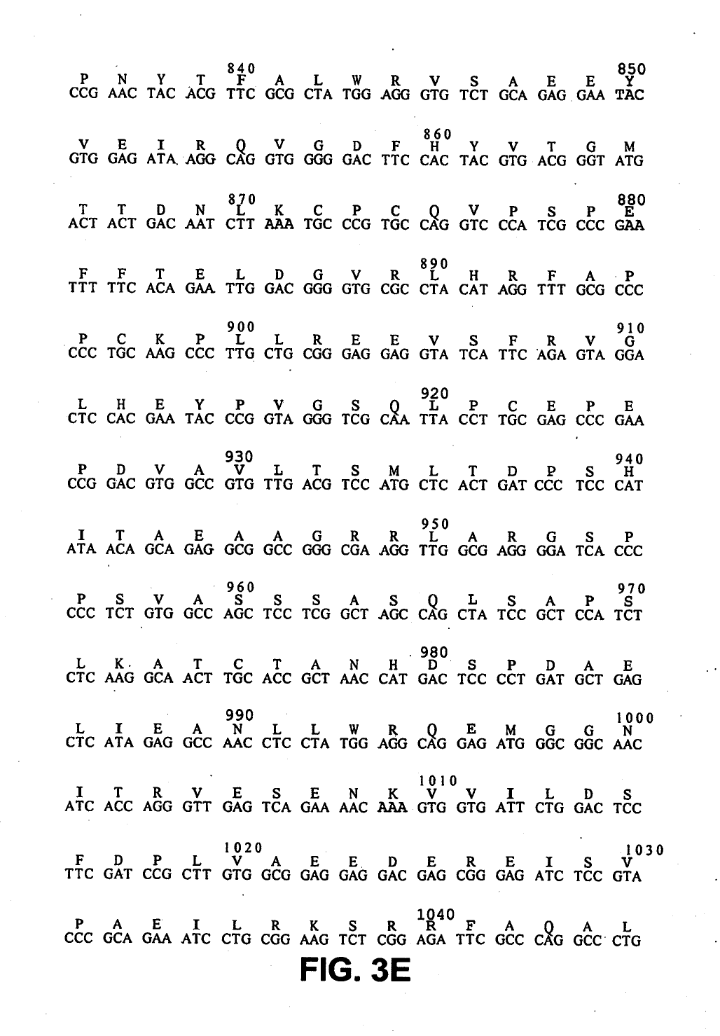

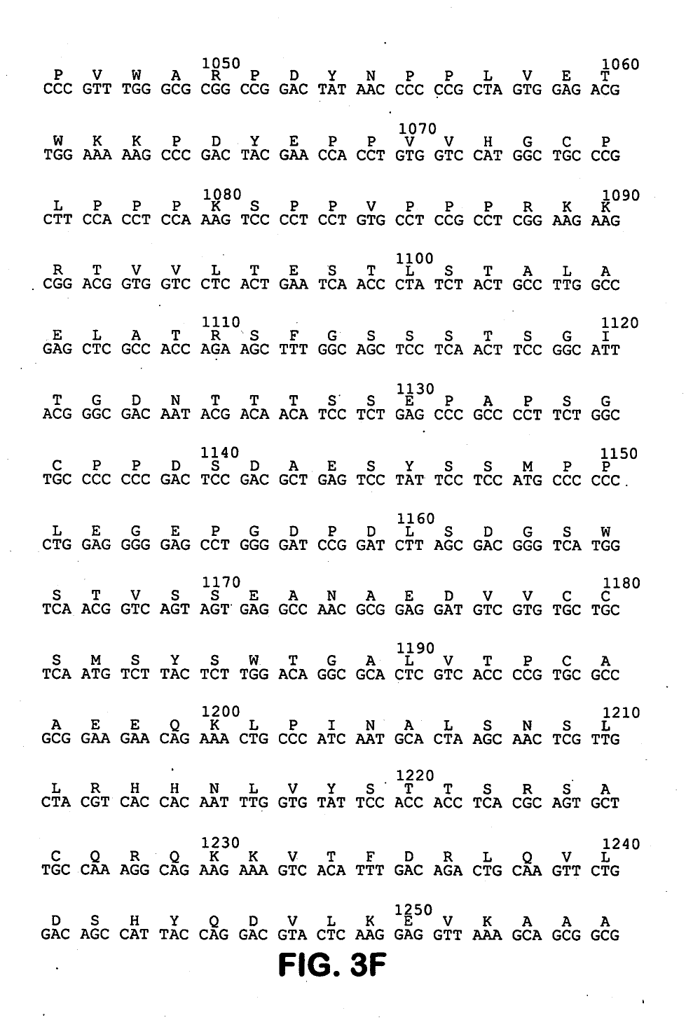

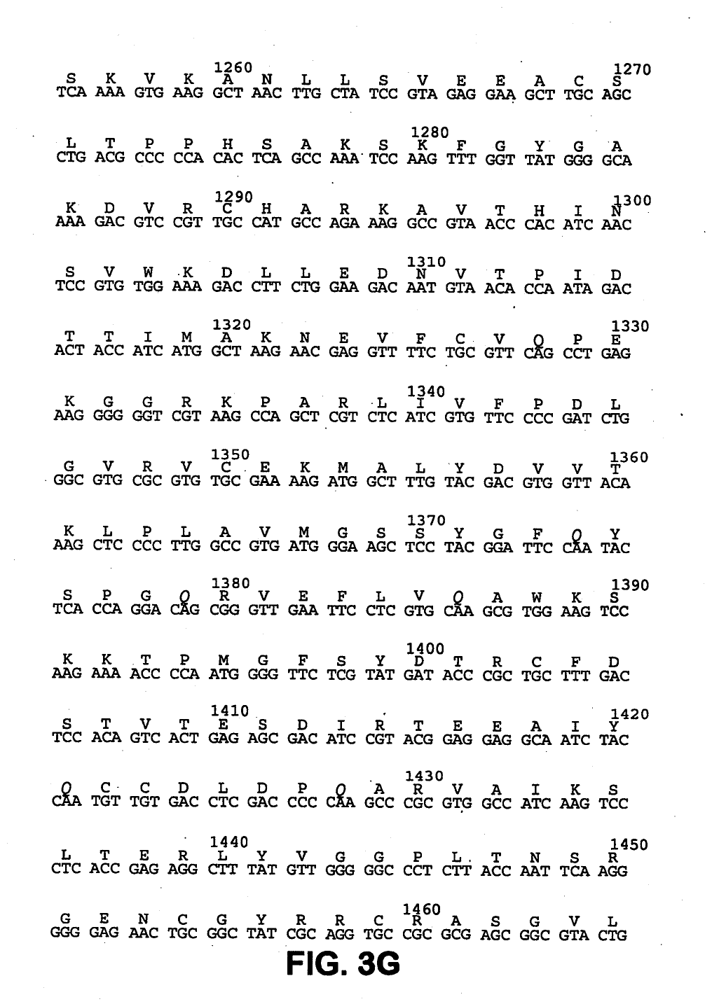

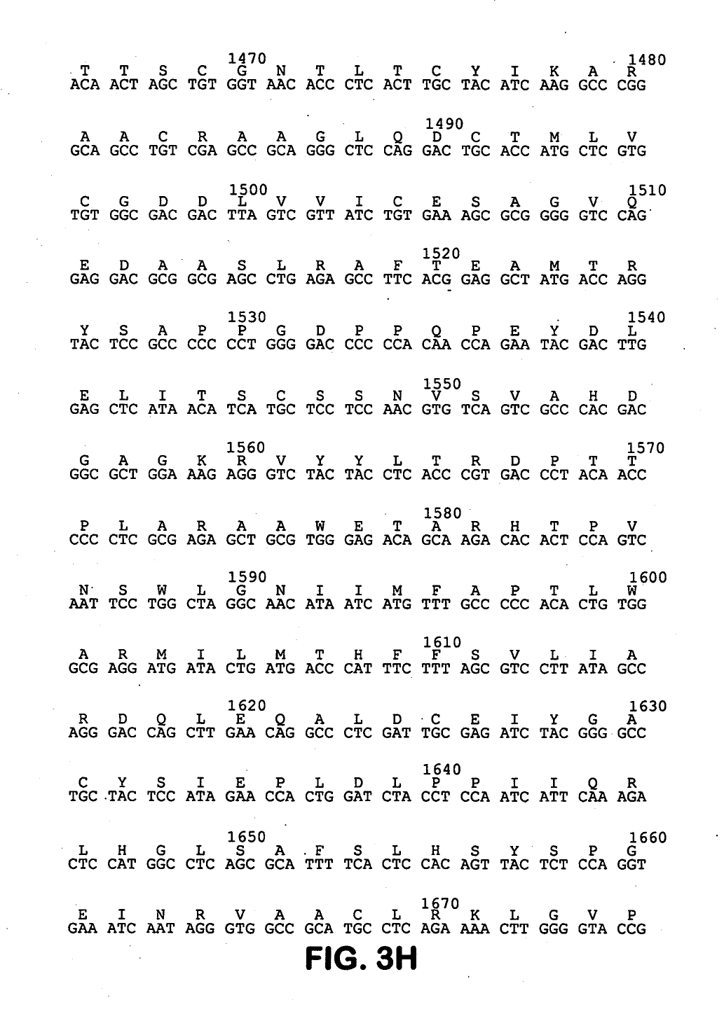

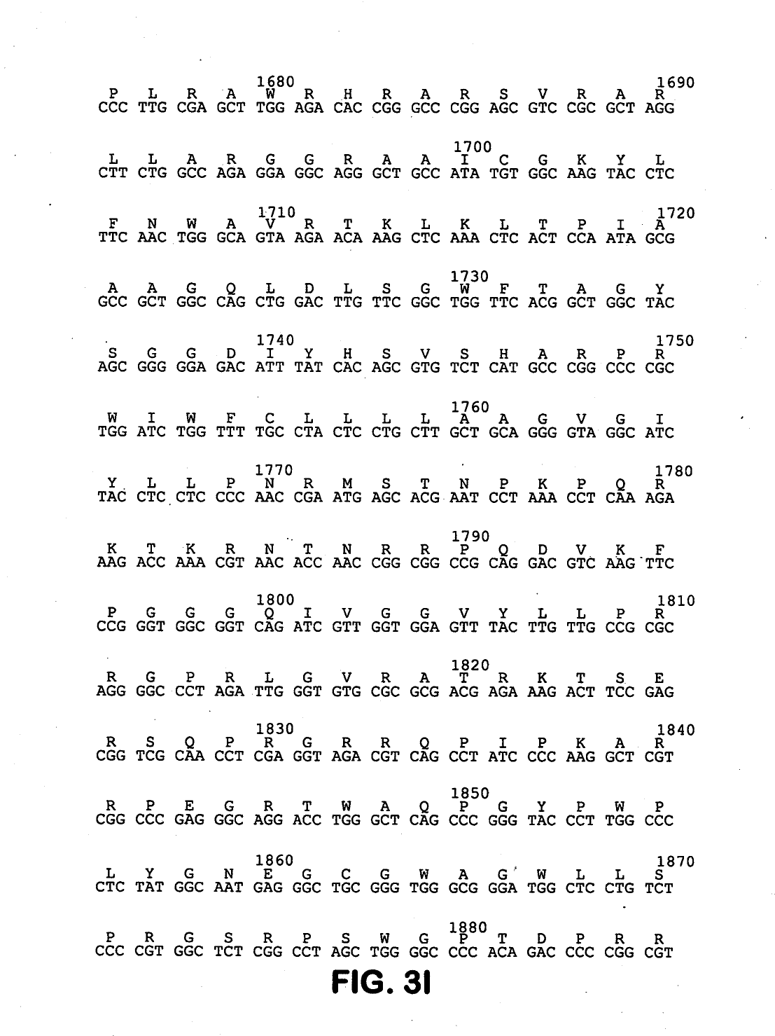

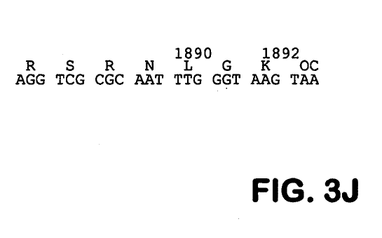

FIG. 3 shows a representative modified NS3 polypeptide, with the NS3 protease domain deleted from the N-terminus and including amino acids 1-121 of Core on the C-terminus.

As explained above, it may be desirable to include polypeptides derived from the core region of the HCV polyprotein in the fusions of the invention. This region occurs at amino acid positions 1-191 of the HCV polyprotein, numbered relative to HCV-1. Either the full-length protein, fragments thereof, such as amino acids 1-160, e.g., amino acids 1-150, 1-140, 1-130, 1-120, for example, amino acids 1-121, 1-122, 1-123 . . . 1-151, etc., or smaller fragments containing epitopes of the full-length protein may be used in the subject fusions, such as those epitopes found between amino acids 10-53, amino acids 10-45, amino acids 67-88, amino acids 120-130, or any of the core epitopes identified in, e.g., Houghton et al., U.S. Pat. No. 5,350,671; Chien et al., Proc. Natl. Acad. Sci. USA (1992) 89:10011-10015; Chien et al., J. Gastroent. Hepatol. (1993) 8:S33-39; Chien et al., International Publication No. WO 93/00365; Chien, D. Y., International Publication No. WO 94/01778; and U.S. Pat. Nos. 6,280,927 and 6,150,087, the disclosures of which are incorporated herein by reference in their entireties. Moreover, a protein resulting from a frameshift in the core region of the polyprotein, such as described in International Publication No. WO 99/63941, may be used. One particularly desirable core polypeptide for use with the present fusions includes the sequence of amino acids depicted at amino acid positions 1772-1892 of FIG. 3. This core polypeptide includes amino acids 1-121 of the HCV polyprotein, with consensus amino acids Arg-9 and Thr-11 (positions 1780 and 1782, respectively, of FIG. 3). FIGS. 5A-5E (SEQ ID NOS:7 and 8) show the DNA and corresponding amino acid sequence of a representative fusion protein that includes a C-terminally truncated NS5 polypeptide with the C-terminus of the NS5 polypeptide fused to this core polypeptide. The C-terminally truncated NS5 polypeptide includes amino acids 1973-2990 of the HCV polyprotein, numbered relative to HCV-1 (see, Choo et al. (1991) Proc. Natl. Acad. Sci. USA 88:2451-2455), (amino acids 1-1018 of SEQ ID NO:7), fused to a core polypeptide as described above that includes amino acids 1-121 of the HCV polyprotein (amino acids 1019-1139 of SEQ ID NO:7).

If a core polypeptide is present, it can occur at the N-terminus, the C-terminus and/or internal to the fusion. Particularly preferred is a core polypeptide on the C-terminus as this allows for the formation of complexes with certain adjuvants, such as ISCOMs, described further below.

Other useful polypeptides in the HCV fusion include T-cell epitopes derived from any of the various regions in the polyprotein. In this regard, E1, E2, p7 and NS2 are known to contain human T-cell epitopes (both CD4+ and CD8+) and including one or more of these epitopes serves to increase vaccine efficacy as well as to increase protective levels against multiple HCV genotypes. Moreover, multiple copies of specific, conserved T-cell epitopes can also be used in the fusions, such as a composite of epitopes from different genotypes.

For example, polypeptides from the HCV E1 and/or E2 regions can be used in the fusions of the present invention. E2 exists as multiple species (Spaete et al., Virol. (1992) 188:819-830; Selby et al., J. Virol. (1996) 70:5177-5182; Grakoui et al., J. Virol. (1993) 67:1385-1395; Tomei et al., J. Virol. (1993) 67:4017-4026) and clipping and proteolysis may occur at the N- and C-termini of the E2 polypeptide. Thus, an E2 polypeptide for use herein may comprise amino acids 405-661, e.g., 400, 401, 402 . . . to 661, as well as polypeptides such as 383 or 384-661, 383 or 384-715, 383 or 384-746, 383 or 384-749 or 383 or 384-809, or 383 or 384 to any C-terminus between 661-809, of an HCV polyprotein, numbered relative to the full-length HCV-1 polyprotein. Similarly, E1 polypeptides for use herein can comprise amino acids 192-326, 192-330, 192-333, 192-360, 192-363, 192-383, or 192 to any C-terminus between 326-383, of an HCV polyprotein.

Immunogenic fragments of E1 and/or E2 which comprise epitopes may be used in the subject fusions. For example, fragments of E1 polypeptides can comprise from about 5 to nearly the full-length of the molecule, such as 6, 10, 25, 50, 75, 100, 125, 150, 175, 185 or more amino acids of an E1 polypeptide, or any integer between the stated numbers. Similarly, fragments of E2 polypeptides can comprise 6, 10, 25, 50, 75, 100, 150, 200, 250, 300, or 350 amino acids of an E2 polypeptide, or any integer between the stated numbers.

For example, epitopes derived from, e.g., the hypervariable region of E2, such as a region spanning amino acids 384-410 or 390-410, can be included in the fusions. A particularly effective E2 epitope to incorporate into an E2 polypeptide sequence is one which includes a consensus sequence derived from this region, such as the consensus sequence Gly-Ser-Ala-Ala-Arg-Thr-Thr-Ser-Gly-Phe-Val-Ser-Leu-Phe-Ala-Pro-Gly-Ala-Lys-Gln-Asn, which represents a consensus sequence for amino acids 390-410 of the HCV type 1 genome. Additional epitopes of E1 and E2 are known and described in, e.g., Chien et al., International Publication No. WO 93/00365.

Moreover, the E1 and/or E2 polypeptides may lack all or a portion of the membrane spanning domain. With E1, generally polypeptides terminating with about amino acid position 370 and higher (based on the numbering of the HCV-1 polyprotein) will be retained by the ER and hence not secreted into growth media. With E2, polypeptides terminating with about amino acid position 731 and higher (also based on the numbering of the HCV-1 polyprotein) will be retained by the ER and not secreted. (See, e.g., International Publication No. WO 96/04301, published Feb. 15, 1996). It should be noted that these amino acid positions are not absolute and may vary to some degree. Thus, the present invention contemplates the use of E1 and/or E2 polypeptides which retain the transmembrane binding domain, as well as polypeptides which lack all or a portion of the transmembrane binding domain, including E1 polypeptides terminating at about amino acids 369 and lower, and E2 polypeptides, terminating at about amino acids 730 and lower. Furthermore, the C-terminal truncation can extend beyond the transmembrane spanning domain towards the N-terminus. Thus, for example, E1 truncations occurring at positions lower than, e.g., 360 and E2 truncations occurring at positions lower than, e.g., 715, are also encompassed by the present invention. All that is necessary is that the truncated E1 and E2 polypeptides remain functional for their intended purpose. However, particularly preferred truncated E1 constructs are those that do not extend beyond about amino acid 300. Most preferred are those terminating at position 360. Preferred truncated E2 constructs are those with C-terminal truncations that do not extend beyond about amino acid position 715. Particularly preferred E2 truncations are those molecules truncated after any of amino acids 715-730, such as 725.

In certain preferred embodiments, the fusion protein comprises a modified NS3, an NS4 (NS4a and NS4b), a C-terminally truncated NS5 and, optionally, a core polypeptide of an HCV (NS3*NS4NS5t or NS3*NS4NS5tCore fusion proteins, also termed “NS3*45t” and “NS3*45tCore” herein). These regions need not be in the order in which they naturally occur in the native HCV polyprotein. Thus, for example, the core polypeptide may be at the N- and/or C-terminus of the fusion. In a particularly preferred embodiment, the NS5t includes amino acids 1973-2990, numbered relative to the full-length HCV-1 polyprotein and the NS3*molecule includes a substitution of Ala for Ser normally found at position 1165, and the regions occur in the following N-terminus to C-terminus order: NS3*NS4NS5t. This fusion can include a core polypeptide at the C-terminus of the molecule. If present, the core polypeptide preferably includes the sequence of amino acids depicted at amino acid positions 1772-1892 of FIG. 3. This core polypeptide includes amino acids 1-121 of the HCV polyprotein, with consensus amino acids Arg-9 and Thr-11 (positions 1780 and 1782, respectively, of FIG. 3).

In another preferred embodiment, the fusion protein described immediately above includes an E2 polypeptide at the N-terminus preceding NS3*. Preferably, the E2 polypeptide is a C-terminally truncated polypeptide and includes amino acids 384-715, numbered relative to the full-length HCV-1 polyprotein. This fusion can also optionally include a core polypeptide as described above.

If desired, the fusion proteins, or the individual components of these proteins, also can contain other amino acid sequences, such as amino acid linkers or signal sequences, as well as ligands useful in protein purification, such as glutathione-S-transferase and staphylococcal protein A.

### Polynucleotides Encoding the Fusion Proteins

Polynucleotides contain less than an entire HCV genome, or alternatively can include the sequence of the entire polyprotein with a C-terminally truncated NS5 domain, as described above. The polynucleotides can be RNA or single- or double-stranded DNA. Preferably, the polynucleotides are isolated free of other components, such as proteins and lipids. The polynucleotides encode the fusion proteins described above, and thus comprise coding sequences for NS5t and at least one other HCV polypeptide from a different region of the HCV polyprotein, such as polypeptides derived from NS2, p7, E1, E2, NS3, NS4, core, etc. Polynucleotides of the invention can also comprise other nucleotide sequences, such as sequences coding for linkers, signal sequences, or ligands useful in protein purification such as glutathione-S-transferase and staphylococcal protein A.

To aid expression yields, it may be desirable to split the polyprotein into fragments for expression. These fragments can be used in combination in compositions as described herein. Alternatively, these fragments can be joined subsequent to expression. Thus, for example, NS3*NS4 can be expressed as one construct and NS5tCore can be expressed as a second construct and the two proteins subsequently fused or added separately to compositions. Similarly, E2NS3*NS4 can be expressed as one construct and NS5tCore expressed as a second construct. It is to be understood that the above combinations are merely representative and any combination of fusions can be expressed separately.

Polynucleotides encoding the various HCV polypeptides can be isolated from a genomic library derived from nucleic acid sequences present in, for example, the plasma, serum, or liver homogenate of an HCV infected individual or can be synthesized in the laboratory, for example, using an automatic synthesizer. An amplification method such as PCR can be used to amplify polynucleotides from either HCV genomic DNA or cDNA encoding therefor.

Polynucleotides can comprise coding sequences for these polypeptides which occur naturally or can be artificial sequences which do not occur in nature. These polynucleotides can be ligated to form a coding sequence for the fusion proteins using standard molecular biology techniques. A polynucleotide encoding these proteins can be introduced into an expression vector which can be expressed in a suitable expression system. A variety of bacterial, yeast, mammalian and insect expression systems are available in the art and any such expression system can be used. Optionally, a polynucleotide encoding these proteins can be translated in a cell-free translation system. Such methods are well known in the art. The proteins also can be constructed by solid phase protein synthesis.

The expression constructs of the present invention, including the desired fusion, or individual expression constructs comprising the individual components of these fusions, may be used for nucleic acid immunization, to stimulate a cellular immune response, using standard gene delivery protocols. Methods for gene delivery are known in the art. See, e.g., U.S. Pat. Nos. 5,399,346, 5,580,859, 5,589,466, incorporated by reference herein in their entireties. Genes can be delivered either directly to the vertebrate subject or, alternatively, delivered ex vivo, to cells derived from the subject and the cells reimplanted in the subject. For example, the constructs can be delivered as plasmid DNA, e.g., contained within a plasmid, such as pBR322, pUC, or ColE1

Additionally, the expression constructs can be packaged in liposomes prior to delivery to the cells. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527.

Liposomal preparations for use with the present invention include cationic (positively charged), anionic (negatively charged) and neutral preparations, with cationic liposomes particularly preferred. Cationic liposomes are readily available. For example, N[1-2,3-dioleyloxy)propyl]-N,N,N-triethyl-ammonium (DOTMA) liposomes are available under the trademark Lipofectin, from GIBCO BRL, Grand Island, N.Y. (See, also, Feigner et al., Proc. Natl. Acad. Sci. USA (1987) 84:7413-7416). Other commercially available lipids include transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA (1978) 75:4194-4198; PCT Publication No. WO 90/11092 for a description of the synthesis of DOTAP (1,2-bis(oleoyloxy)-3-(trimethylammonio)propane) liposomes. The various liposome-nucleic acid complexes are prepared using methods known in the art. See, e.g., Straubinger et al., in METHODS OF IMMUNOLOGY (1983), Vol. 101, pp. 512-527; Szoka et al., Proc. Natl. Acad. Sci. USA (1978) 75:4194-4198; Papahadjopoulos et al., Biochim. Biophys. Acta (1975) 394:483; Wilson et al., Cell (1979) 17:77); Deamer and Bangham, Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); Enoch and Strittmatter, Proc. Natl. Acad. Sci. USA (1979) 76:145); Fraley et al., J. Biol. Chem. (1980) 255:10431; Szoka and Papahadjopoulos, Proc. Natl. Acad. Sci. USA (1978) 75:145; and Schaefer-Ridder et al., Science (1982) 215:166.

The DNA can also be delivered in cochleate lipid compositions similar to those described by Papahadjopoulos et al., Biochem. Biophys. Acta. (1975) 394:483-491. See, also, U.S. Pat. Nos. 4,663,161 and 4,871,488.

A number of viral based systems have been developed for gene transfer into mammalian cells. For example, retroviruses provide a convenient platform for gene delivery systems, such as murine sarcoma virus, mouse mammary tumor virus, Moloney murine leukemia virus, and Rous sarcoma virus. A selected gene can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman, BioTechniques (1989) 7:980-990; Miller, A. D., Human Gene Therapy (1990) 1:5-14; Scarpa et al., Virology (1991) 180:849-852; Burns et al., Proc. Natl. Acad. Sci. USA (1993) 90:8033-8037; and Boris-Lawrie and Temin, Cur. Opin. Genet. Develop. (1993) 3:102-109. Briefly, retroviral gene delivery vehicles of the present invention may be readily constructed from a wide variety of retroviruses, including for example, B, C, and D type retroviruses as well as spumaviruses and lentiviruses such as FIV, HIV, HIV-1, HIV-2 and SW (see RNA Tumor Viruses, Second Edition, Cold Spring Harbor Laboratory, 1985). Such retroviruses may be readily obtained from depositories or collections such as the American Type Culture Collection (“ATCC”; 10801 University Blvd., Manassas, Va. 20110-2209), or isolated from known sources using commonly available techniques.

A number of adenovirus vectors have also been described, such as adenovirus Type 2 and Type 5 vectors. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476).

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as but not limited to vectors derived from the Sindbis and Semliki Forest viruses, VEE, will also find use as viral vectors for delivering the gene of interest. For a description of Sindbis-virus derived vectors useful for the practice of the instant methods, see, Dubensky et al., J. Virol. (1996) 70:508-519; and International Publication Nos. WO 95/07995 and WO 96/17072.

Other vectors can be used, including but not limited to adeno-associated virus vectors, simian virus 40 and cytomegalovirus. Bacterial vectors, such as Salmonella ssp. Yersinia enterocolitica, Shigella spp., Vibrio cholerae, Mycobacterium strain BCG, and Listeria monocytogenes can be used. Minichromosomes such as MC and MC1, bacteriophages, cosmids (plasmids into which phage lambda cos sites have been inserted) and replicons (genetic elements that are capable of replication under their own control in a cell) can also be used.

The expression constructs may also be encapsulated, adsorbed to, or associated with, particulate carriers. Such carriers present multiple copies of a selected molecule to the immune system and promote trapping and retention of molecules in local lymph nodes. The particles can be phagocytosed by macrophages and can enhance antigen presentation through cytokine release. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

A wide variety of other methods can be used to deliver the expression constructs to cells. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, liposomes, peptoid delivery, or microinjection. See, e.g., Sambrook et al., supra, for a discussion of techniques for transforming cells of interest; and Felgner, P. L., Advanced Drug Delivery Reviews (1990) 5:163-187, for a review of delivery systems useful for gene transfer. One particularly effective method of delivering DNA using electroporation is described in International Publication No. WO/0045823.

Additionally, biolistic delivery systems employing particulate carriers such as gold and tungsten, are especially useful for delivering the expression constructs of the present invention. The particles are coated with the construct to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744.

### Compositions Comprising Fusion Proteins or Polynucleotides

The invention also provides compositions comprising the fusion proteins or polynucleotides. The compositions may be used to stimulate an immunological response, as defined above. The compositions may include one or more fusions, so long as one of the fusions includes a C-terminally truncated NS5 domain as described herein. Compositions of the invention may also comprise a pharmaceutically acceptable carrier. The carrier should not itself induce the production of antibodies harmful to the host. Pharmaceutically acceptable carriers are well known to those in the art. Such carriers include, but are not limited to, large, slowly metabolized, macromolecules, such as proteins, polysaccharides such as latex functionalized sepharose, agarose, cellulose, cellulose beads and the like, polylactic acids, polyglycolic acids, polymeric amino acids such as polyglutamic acid, polylysine, and the like, amino acid copolymers, and inactive virus particles.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art. Compositions of the invention can also contain liquids or excipients, such as water, saline, glycerol, dextrose, ethanol, or the like, singly or in combination, as well as substances such as wetting agents, emulsifying agents, or pH buffering agents. The proteins or polynucleotides of the invention can also be adsorbed to, entrapped within or otherwise associated with liposomes and particulate carriers such as PLG. Liposomes and other particulate carriers are described above.

If desired, co-stimulatory molecules which improve immunogen presentation to lymphocytes, such as B7-1 or B7-2, or cytokines, lymphokines, and chemokines, including but not limited to cytokines such as IL-2, modified IL-2 (cys125 to ser125), GM-CSF, IL-12, γ-interferon, IP-10, MIP1β, FLP-3, ribavirin and RANTES, may be included in the composition. Optionally, adjuvants can also be included in a composition. Adjuvants which can be used include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (PCT Publ. No. WO 90/14837), containing 5% Squalene, 0.5% TWEEN 80, and 0.5% SPAN 85 (optionally containing various amounts of MTP-PE), formulated into submicron particles using a microfluidizer such as Model 110Y microfluidizer (Microfluidics, Newton, Mass.), (b) SAF, containing 10% Squalane, 0.4% TWEEN 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% TWEEN 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as QS21 or Stimulon™ (Cambridge Bioscience, Worcester, Mass.) may be used or particles generated therefrom such as ISCOMs (immunostimulating complexes), which ISCOMs may be devoid of additional detergent (see, e.g., International Publication No. WO 00/07621); (4) Complete Freunds Adjuvant (CFA) and Incomplete Freunds Adjuvant (IFA); (5) cytokines, such as interleukins, such as IL-1, IL-2, IL-4, IL-5, IL-6, IL-7, IL-12 etc. (see, e.g., International Publication No. WO 99/44636), interferons, such as gamma interferon, macrophage colony stimulating factor (M-CSF), tumor necrosis factor (TNF), etc.; (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); (7) monophosphoryl lipid A (MPL) or 3-O-deacylated MPL (3dMPL) (see, e.g., GB 2220221; EPA 0689454), optionally in the substantial absence of alum (see, e.g., International Publication No. WO 00/56358); (8) combinations of 3dMPL with, for example, QS21 and/or oil-in-water emulations (see, e.g., EPA 0835318; EPA 0735898; EPA 0761231); (9) a polyoxyethylene ether or a polyoxyethylene ester (see, e.g., International Publication No. WO 99/52549); (10) an immunostimulatory oligonucleotide such as a CpG oligonucleotide, or a saponin and an immunostimulatory oligonucleotide, such as a CpG oligonucleotide (see, e.g., International Publication No. WO 00/62800); (11) an immunostimulant and a particle of a metal salt (see, e.g., International Publication No. WO 00/23105); (12) a saponin and an oil-in-water emulsion (see, e.g., International Publication No. WO 99/11241; (13) a saponin (e.g., QS21)+3dMPL+IL-12 (optionally+a sterol) (see, e.g., International Publication No. WO 98/57659); (14) the MPL derivative RC529; and (15) other substances that act as immunostimulating agents to enhance the effectiveness of the composition. Alum and MF59 are preferred.

As mentioned above, muramyl peptides include, but are not limited to, N-acetyl-muramyl-L-threonyl-D-isoglutamine (thr-MDP), -acetyl-normuramyl-L-alanyl-D-isoglutamine (CGP 11637, referred to nor-MDP), N-acetylmuramyl-L-alanyl-D-isoglutaminyl-L-alanine-2-(1′-2′-dipalmitoyl-sn-glycero-3-h ydroxyphosphoryloxy)-ethylamine (CGP 19835A, referred to as MTP-PE), etc.

Moreover, the fusion protein can be adsorbed to, or entrapped within, an ISCOM. Classic ISCOMs are formed by combination of cholesterol, saponin, phospholipid, and immunogens. Generally, immunogens (usually with a hydrophobic region) are solubilized in detergent and added to the reaction mixture, whereby ISCOMs are formed with the immunogen incorporated therein. ISCOM matrix compositions are formed identically, but without viral proteins. Proteins with high positive charge may be electrostatically bound in the ISCOM particles, rather than through hydrophobic forces. For a more detailed general discussion of saponins and ISCOMs, and methods of formulating ISCOMs, see Barr et al. (1998) Adv. Drug Delivery Reviews 32:247-271 (1998).

ISCOMs for use with the present invention are produced using standard techniques, well known in the art, and are described in e.g., U.S. Pat. Nos. 4,981,684, 5,178,860, 5,679,354 and 6,027,732; European Publ. Nos. EPA 109,942; 180,564 and 231,039; Coulter et al. (1998) Vaccine 16:1243. Typically, the term “ISCOM” refers to immunogenic complexes formed between glycosides, such as triterpenoid saponins (particularly Quil A), and antigens which contain a hydrophobic region. See, e.g., European Publ. Nos. EPA 109,942 and 180,564. In this embodiment, the HCV fusions (usually with a hydrophobic region) are solubilized in detergent and added to the reaction mixture, whereby ISCOMs are formed with the fusions incorporated therein. The HCV polypeptide ISCOMs are readily made with HCV polypeptides which show amphipathic properties. However, proteins and peptides which lack the desirable hydrophobic properties may be incorporated into the immunogenic complexes after coupling with peptides having hydrophobic amino acids, fatty acid radicals, alkyl radicals and the like.

As explained in European Publ. No. EPA 231,039, the presence of antigen is not necessary in order to form the basic ISCOM structure (referred to as a matrix or ISCOMATRIX), which may be formed from a sterol, such as cholesterol, a phospholipid, such as phosphatidylethanolamine, and a glycoside, such as Quil A. Thus, the HCV fusion of interest, rather than being incorporated into the matrix, is present on the outside of the matrix, for example adsorbed to the matrix via electrostatic interactions. For example, HCV fusions with high positive charge may be electrostatically bound to the ISCOM particles, rather than through hydrophobic forces. For a more detailed general discussion of saponins and ISCOMs, and methods of formulating ISCOMs, see Barr et al. (1998) Adv. Drug Delivery Reviews 32:247-271 (1998).

The ISCOM matrix may be prepared, for example, by mixing together solubilized sterol, glycoside and (optionally) phospholipid. If phospholipids are not used, two dimensional structures are formed. See, e.g., European Publ. No. EPA 231,039. The term “ISCOM matrix” is used to refer to both the 3-dimensional and 2-dimensional structures. The glycosides to be used are generally glycosides which display amphipathic properties and comprise hydrophobic and hydrophilic regions in the molecule. Preferably saponins are used, such as the saponin extract from Quillaja saponaria Molina and Quil A. Other preferred saponins are aescine from Aesculus hippocastanum (Patt et al. (1960) Arzneimittelforschung 10:273-275 and sapoalbin from Gypsophilla struthium (Vochten et al. (1968)J. Pharm. Belg. 42:213-226.

In order to prepare the ISCOMs, glycosides are used in at least a critical micelle-forming concentration. In the case of Quil A, this concentration is about 0.03% by weight. The sterols used to produce ISCOMs may be known sterols of animal or vegetable origin, such as cholesterol, lanosterol, lumisterol, stigmasterol and sitosterol. Suitable phospholipids include phosphatidylcholine and phosphatidylethanolamine. Generally, the molar ratio of glycoside (especially when it is Quil A) to sterol (especially when it is cholesterol) to phospholipid is 1:1:0-1, +20% (preferably not more than ±10%) for each figure. This is equivalent to a weight ratio of about 5:1 for the Quil A:cholesterol.

A solubilizing agent may also be present and may be, for example a detergent, urea or guanidine. Generally, a non-ionic, ionic or zwitter-ionic detergent or a cholic acid based detergent, such as sodium desoxycholate, cholate and CTAB (cetyltriammonium bromide), can be used for this purpose. Examples of suitable detergents include, but are not limited to, octylglucoside, nonyl N-methyl glucamide or decanoyl N-methyl glucamide, alkylphenyl polyoxyethylene ethers such as a polyethylene glycol p-isooctyl-phenylether having 9 to 10 oxyethylene groups (commercialized under the trade name TRITON X-100R™), acylpolyoxyethylene esters such as acylpolyoxyethylene sorbitane esters (commercialized under the trade name TWEEN 20™, TWEEN 80™, and the like). The solubilizing agent is generally removed for formation of the ISCOMs, such as by ultrafiltration, dialysis, ultracentrifugation or chromatography, however, in certain methods, this step is unnecessary. (See, e.g., U.S. Pat. No. 4,981,684).

Generally, the ratio of glycoside, such as QuilA, to HCV fusion by weight is in the range of 5:1 to 0.5:1. Preferably the ratio by weight is approximately 3:1 to 1:1, and more preferably the ratio is 2:1.

Once the ISCOMs are formed, they may be formulated into compositions and administered to animals, as described herein. If desired, the solutions of the immunogenic complexes obtained may be lyophilized and then reconstituted before use.

The NS5 fusion proteins and compositions including the proteins or polynucleotides described above, can be used in combination with other HCV immunogenic proteins, and/or compositions comprising the same. For example, the NS5 fusion proteins can be used in combination with any of the various HCV immunogenic proteins derived from one or more of the regions of the HCV polyprotein described in Table 1. One particular HCV antigen for use with the subject fusions and/or composition comprising the NS5 fusion, is an HCV E1E2 antigen. HCV E1E2 antigens are known, including complexes of HCV E1 with HCV E2, optionally containing part or all of the p7 region, such as HCV E1E2 complexes as described in PCT Publication No. WO 03/002065, incorporated herein by reference in its entirety. The additional HCV immunogenic proteins can be provided in compositions with excipients, adjuvants, immunstimulatory molecules and the like, as described above. For example, the E1E2 complexes can be provided in compositions that include a submicron oil-in-water emulsion such as MF59 and/or oligonucleotides containing immunostimulatory nucleic acid sequences (ISS), such as CpY, CpR and unmethylated CpG motifs (a cytosine followed by guanosine and linked by a phosphate bond). Such compositions are described in detail in PCT Publication No. WO 03/002065, incorporated herein by reference in its entirety. Moreover, the

Thus, it is readily apparent that the compositions of the present invention may be administered in conjunction with a number of immunoregulatory agents and will usually include an adjuvant. Such agents and adjuvants for use with the compositions include, but are not limited to, any of those substances described above, as well as one or more of the following set forth below.

A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design (1995) eds. Powell & Newman. ISBN: 030644867X. Plenum), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (PCT Publication No. WO00/23105).

Aluminum salts may be included in compositions of the invention such that the dose of Al³⁺ is between 0.2 and 1.0 mg per dose. In one embodiment, the aluminum-based adjuvant for use in the present compositions is alum (aluminum potassium sulfate (AlK(SO₄)₂)), or an alum derivative, such as that formed in situ by mixing an antigen in phosphate buffer with alum, followed by titration and precipitation with a base such as ammonium hydroxide or sodium hydroxide.

Another aluminum-based adjuvant for use in vaccine formulations of the present invention is aluminum hydroxide adjuvant (Al(OH)₃) or crystalline aluminum oxyhydroxide (AlOOH), which is an excellent adsorbant, having a surface area of approximately 500 m²/g. Alternatively, aluminum phosphate adjuvant (AlPO₄) or aluminum hydroxyphosphate, which contains phosphate groups in place of some or all of the hydroxyl groups of aluminum hydroxide adjuvant is provided. Preferred aluminum phosphate adjuvants provided herein are amorphous and soluble in acidic, basic and neutral media.

In another embodiment, the adjuvant for use with the present compositions comprises both aluminum phosphate and aluminum hydroxide. In a more particular embodiment thereof, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. More particularly, aluminum salts may be present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (iep=4) adsorbs lysozyme, but not albumin at pH 7.4. Should albumin be the target, aluminum hydroxide adjuvant would be selected (iep 11.4). Alternatively, pretreatment of aluminum hydroxide with phosphate lowers its isoelectric point, making it a preferred adjuvant for more basic antigens.

B. Oil Emulsions

Oil emulsion compositions suitable for use as adjuvants in the compositions include squalene-water emulsions. Particularly preferred adjuvants are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphosphoryloxy)-ethylamine (MTP-PE), for example, the submicron oil-in-water emulsion known as “MF59” (International Publication No. WO90/14837; U.S. Pat. Nos. 6,299,884 and 6,451,325, and Ott et al., “MF59—Design and Evaluation of a Safe and Potent Adjuvant for Human Vaccines” in Vaccine Design The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO90/14837 and U.S. Pat. Nos. 6,299,884 and

6,451,325.

Complete Freund's adjuvant (CFA) and incomplete Freund's adjuvant (IFA) may also be used as adjuvants in the subject compositions.

C. Saponin Formulations

Saponin formulations, may also be used as adjuvants in the compositions. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see, PCT Publication No. WO96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO96/11711 and WO96/33739. Optionally, the ISCOMS may be devoid of (an) additional detergent(s). See WO00/07621.

A review of the development of saponin-based adjuvants can be found in Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

D. Virosomes and Virus Like Particles (VLPs)

Virosomes and Virus Like Particles (VLPs) can also be used as adjuvants with the present compositions. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO03/024480, WO03/024481, and Niikura et al., “Chimeric Recombinant Hepatitis E Virus-Like Particles as an Oral Vaccine Vehicle Presenting Foreign Epitopes”, Virology (2002) 293:273-280; Lenz et al., “Papillomarivurs-Like Particles Induce Acute Activation of Dendritic Cells”, Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338; and Gerber et al., “Human Papillomavrisu Virus-Like Particles Are Efficient Oral Immunogens when Coadministered with Escherichia coli Heat-Labile Entertoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B10-B16. Immunopotentiating reconstituted influenza virosomes (IRIV) are used as the subunit antigen delivery system in the intranasal trivalent INFLEXAL™ product {Mischler & Metcalfe (2002)Vaccine 20 Suppl 5:B17-23} and the INFLUVAC PLUS™ product.

E. Bacterial or Microbial Derivatives

Adjuvants suitable for use in the present compositions include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

(2) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.

(3) Immunostimulatory Oligonucleotides Immunostimulatory oligonucleotides suitable for use as adjuvants include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See, Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See, Kandimalla, et al., “Toll-like receptor 9: modulation of recognition and cytokine induction by novel synthetic CpG DNAs”, Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068; Krieg, “From A to Z on CpG”, TRENDS in Immunology (2002) 23(2): 64-65 and WO01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31(part 3):664-658; Bhagat et al., “CpG penta- and hexadeoxyribonucleotides as potent immunomodulatory agents” BBRC (2003) 300:853-861 and WO03/035836.

(4) ADP-Ribosylating Toxins and Detoxified Derivatives Thereof.

Bacterial ADP-ribosylating toxins and detoxified derivatives thereof may be used as adjuvants in the compositions. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon, et al., “The LTR72Mutant of Heat-Labile Enterotoxin of Escherichia coli Enhances the Ability of Peptide Antigens to Elicit CD4+T Cells and Secrete Gamma Interferon after Coapplication onto Bare Skin”, Infection and Immunity (2002) 70(6):3012-3019; Pizza, et al., “Mucosal vaccines: non toxic derivatives of LT and CT as mucosal adjuvants”, Vaccine (2001) 19:2534-2541; Pizza, et al., “LTK63 and LTR72, two mucosal adjuvants ready for clinical trials” Int. J. Med. Microbiol (2000) 290(4-5):455-461; Scharton-Kersten et al., “Transcutaneous Immunization with Bacterial ADP-Ribosylating Exotoxins, Subunits and Unrelated Adjuvants”, Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216; Peppoloni et al., “Mutants of the Escherichia coli heat-labile enterotoxin as safe and strong adjuvants for intranasal delivery of vaccines”, Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol. (1995) 15(6):1165-1167.

F. Bioadhesives and Mucoadhesives

Bioadhesives and mucoadhesives may also be used as adjuvants in the subject compositions. Suitable bioadhesives include esterified hyaluronic acid microspheres (Singh et al. (2001) J. Cont. Rele. 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the compositions. See, e.g., WO99/27960.

G. Microparticles

Microparticles may also be used as adjuvants in the compositions. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

H. Liposomes

Examples of liposome formulations suitable for use as adjuvants are described in U.S. Pat. No. 6,090,406, U.S. Pat. No. 5,916,588, and EP 0 626 169.

I. Polyoxyethylene Ether and Polyoxyethylene Ester Formulations

Adjuvants suitable for use in the compositions include polyoxyethylene ethers and polyoxyethylene esters. See, e.g., WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109-115 and Payne et al., “Protein Release from Polyphosphazene Matrices”, Adv. Drug. Delivery Review (1998) 31(3):185-196.

K. Muramyl Peptides

Examples of muramyl peptides suitable for use as adjuvants include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

L. Imidazoquinoline. Compounds Examples of imidazoquinoline compounds suitable for use as adjuvants in the compositions include Imiquimod and its analogues, described further in Stanley, “Imiquimod and the imidazoquinolines: mechanism of action and therapeutic potential” Clin Exp Dermatol (2002) 27(7):571-577; Jones, “Resiquimod 3M”, Curr Opin Investig Drugs (2003) 4(2):214-218; and U.S. Pat. Nos. 4,689,338, 5,389,640, 5,268,376, 4,929,624, 5,266,575, 5,352,784, 5,494,916, 5,482,936, 5,346,905, 5,395,937, 5,238,944, and 5,525,612.

M. Thiosemicarbazone Compounds

Examples of thiosemicarbazone compounds, as well as methods of formulating, manufacturing, and screening for compounds all suitable for use as adjuvants in the compositions include those described in WO04/60308. The thiosemicarbazones are particularly effective in the stimulation of human peripheral blood mononuclear cells for the production of cytokines, such as TNF-α.

N. Tryptanthrin Compounds

Examples of tryptanthrin compounds, as well as methods of formulating, manufacturing, and screening for compounds all suitable for use as adjuvants in the compositions include those described in WO04/64759. The tryptanthrin compounds are particularly effective in the stimulation of human peripheral blood mononuclear cells for the production of cytokines, such as TNF-α.

O. Human Immunomodulators

Human immunomodulators suitable for use as adjuvants in the compositions include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

The compositions may also comprise combinations of aspects of one or more of the adjuvants identified above. For example, the following adjuvant compositions may be used in the invention:

(1) a saponin and an oil-in-water emulsion (WO99/11241);

(2) a saponin (e.g., QS21)+a non-toxic LPS derivative (e.g. 3dMPL) (see WO94/00153);

(3) a saponin (e.g., QS21)+a non-toxic LPS derivative (e.g. 3dMPL)+a cholesterol;

(4) a saponin (e.g. QS21)+3dMPL+IL-12 (optionally+a sterol) (WO98/57659);

(5) combinations of 3dMPL with, for example, QS21 and/or oil-in-water emulsions (See European patent applications 0835318, 0735898 and 0761231);

(6) SAF, containing 10% Squalane, 0.4% Tween 80, 5% pluronic-block polymer L121, and thr-MDP, either microfluidized into a submicron emulsion or vortexed to generate a larger particle size emulsion.

(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and

(8) one or more mineral salts (such as an aluminum salt)+a non-toxic derivative of LPS (such as 3dPML).

(9) one or more mineral salts (such as an aluminum salt)+an immunostimulatory oligonucleotide (such as a nucleotide sequence including a CpG motif).

Aluminum salts and MF59 are preferred adjuvants for use with injectable vaccines. Bacterial toxins and bioadhesives are preferred adjuvants for use with mucosally-delivered vaccines, such as nasal vaccines.

The contents of all of the above cited patents, patent applications and journal articles are incorporated by reference as if set forth fully herein.

### Methods of Producing HCV-Specific Antibodies

The HCV fusion proteins can be used to produce HCV-specific polyclonal and monoclonal antibodies. HCV-specific polyclonal and monoclonal antibodies specifically bind to HCV antigens. Polyclonal antibodies can be produced by administering the fusion protein to a mammal, such as a mouse, a rabbit, a goat, or a horse. Serum from the immunized animal is collected and the antibodies are purified from the plasma by, for example, precipitation with ammonium sulfate, followed by chromatography, preferably affinity chromatography. Techniques for producing and processing polyclonal antisera are known in the art.

Monoclonal antibodies directed against HCV-specific epitopes present in the fusion proteins can also be readily produced. Normal B cells from a mammal, such as a mouse, immunized with an HCV fusion protein, can be fused with, for example, HAT-sensitive mouse myeloma cells to produce hybridomas. Hybridomas producing HCV-specific antibodies can be identified using RIA or ELISA and isolated by cloning in semi-solid agar or by limiting dilution. Clones producing HCV-specific antibodies are isolated by another round of screening.

Antibodies, either monoclonal and polyclonal, which are directed against HCV epitopes, are particularly useful for detecting the presence of HCV or HCV antigens in a sample, such as a serum sample from an HCV-infected human. An immunoassay for an HCV antigen may utilize one antibody or several antibodies. An immunoassay for an HCV antigen may use, for example, a monoclonal antibody directed towards an HCV epitope, a combination of monoclonal antibodies directed towards epitopes of one HCV polypeptide, monoclonal antibodies directed towards epitopes of different HCV polypeptides, polyclonal antibodies directed towards the same HCV antigen, polyclonal antibodies directed towards different HCV antigens, or a combination of monoclonal and polyclonal antibodies. Immunoassay protocols may be based, for example, upon competition, direct reaction, or sandwich type assays using, for example, labeled antibody. The labels may be, for example, fluorescent, chemiluminescent, or radioactive.

The polyclonal or monoclonal antibodies may further be used to isolate HCV particles or antigens by immunoaffinity columns. The antibodies can be affixed to a solid support by, for example, adsorption or by covalent linkage so that the antibodies retain their immunoselective activity. Optionally, spacer groups may be included so that the antigen binding site of the antibody remains accessible. The immobilized antibodies can then be used to bind HCV particles or antigens from a biological sample, such as blood or plasma. The bound HCV particles or antigens are recovered from the column matrix by, for example, a change in pH.

HCV-Specific T cells

HCV-specific T cells that are activated by the above-described fusions, including the NS3*NS4NS5t fusion protein or E2NS3*NS4NS5t fusion protein, with or without a core polypeptide, as well as any of the other various fusions described herein, expressed in vivo or in vitro, preferably recognize an epitope of an HCV polypeptide such as an NS2, p′7, E1, E2, NS3, NS4, NS5a or NS5b polypeptide, including an epitope of a fusion of one or more of these peptides with an NS5t, with or without a core polypeptide. HCV-specific T cells can be CD8⁺ or CD4⁺.

HCV-specific CD8⁺T cells can be cytotoxic T lymphocytes (CTL) which can kill HCV-infected cells that display any of these epitopes complexed with an MHC class I molecule. HCV-specific CD8⁺T cells can be detected by, for example, ⁵¹Cr release assays (see the examples). ⁵¹Cr release assays measure the ability of HCV-specific CD8⁺T cells to lyse target cells displaying one or more of these epitopes. HCV-specific CD8⁺T cells which express antiviral agents, such as IFN-γ, are also contemplated herein and can also be detected by immunological methods, preferably by intracellular staining for IFN-γ or like cytokine after in vitro stimulation with one or more of the HCV polypeptides, such as but not limited to an E2, NS3, NS4, NS5a, or NS5b polypeptide (see the examples).

HCV-specific CD4⁺ cells activated by the above-described fusions, such as but not limited to an NS3*NS4NS5t fusion protein or an E2NS3*NS4NS5t fusion protein, with or without a core polypeptide, expressed in vivo or in vitro, preferably recognize an epitope of an HCV polypeptide, such as but not limited to an NS2, p′7, E1, E2, NS3, NS4, NS5a, or NS5b polypeptide, including an epitope of fusions thereof, bound to an MHC class II molecule on an HCV-infected cell and proliferate in response to stimulating, e.g., NS3*NS4NS5t or E2NS3*NS4NS5t fusion protein, with or without a core polypeptide. HCV-specific CD4⁺T cells can be detected by a lymphoproliferation assay (see the examples). Lymphoproliferation assays measure the ability of HCV-specific CD4⁺T cells to proliferate in response to, e.g., an NS2, p7, E1, E2, NS3, an NS4, an NS5a, and/or an NS5b epitope.

Methods of Activating HCV-Specific T Cells.

The HCV fusion proteins or polynucleotides can be used to activate HCV-specific T cells either in vitro or in vivo. Activation of HCV-specific T cells can be used, inter alia, to provide model systems to optimize CTL responses to HCV and to provide prophylactic or therapeutic treatment against HCV infection. For in vitro activation, proteins are preferably supplied to T cells via a plasmid or a viral vector, such as an adenovirus vector, as described above.

Polyclonal populations of T cells can be derived from the blood, and preferably from peripheral lymphoid organs, such as lymph nodes, spleen, or thymus, of mammals that have been infected with an HCV. Preferred mammals include mice, chimpanzees, baboons, and humans. The HCV serves to expand the number of activated HCV-specific T cells in the mammal. The HCV-specific T cells derived from the mammal can then be restimulated in vitro by adding an HCV fusion protein as described herein, such as but not limited to an HCV NS3*NS4NS5t fusion protein or an E2NS3*NS4NS5t fusion protein, with or without a core polypeptide, to the T cells. The HCV-specific T cells can then be tested for, inter alia, proliferation, the production of IFN-γ, and the ability to lyse target cells displaying HCV epitopes in vitro.

In a lymphoproliferation assay (see Example 6), HCV-activated CD4⁺T cells proliferate when cultured with an HCV polypeptide, such as but not limited to an NS3, NS4, NS5a, NS5b, NS3NS4NS5, or E2NS3NS4NS5 epitopic peptide, but not in the absence of an epitopic peptide. Thus, particular HCV epitopes, such as NS2, p7, E1, E2, NS3, NS4, NS5a, NS5b, and fusions of these epitopes, such as but not limited to NS3NS4NS5 and E2NS3NS4NS5 epitopes that are recognized by HCV-specific CD4⁺T cells can be identified using a lymphoproliferation assay.

Similarly, detection of IFN-γ in HCV-specific CD4+ and/or CD8⁺T cells after in vitro stimulation with the above-described fusion proteins, can be used to identify, for example, fusion protein epitopes, such as but not limited to epitopes of NS2, p7, E1, E2, NS3, NS4, NS5a, NS5b, and fusions of these epitopes, such as but not limited to NS3NS4NS5, and E2NS3NS4NS5 epitopes that are particularly effective at stimulating CD4+ and/or CD8⁺T cells to produce IFN-γ (see Example 5).

Further, ⁵¹Cr release assays are useful for determining the level of CTL response to HCV. See Cooper et al. Immunity 10:439-449. For example, HCV-specific CD8⁺T cells can be derived from the liver of an HCV infected mammal. These T cells can be tested in ⁵¹Cr release assays against target cells displaying, e.g., E2NS3NS4NS5 or NS3NS4NS5 epitopes. Several target cell populations expressing different NS3NS4NS5 or E2NS3NS4NS5 epitopes can be constructed so that each target cell population displays different epitopes of NS3NS4NS5 or E2NS3NS4NS5. The HCV-specific CD8+ cells can be assayed against each of these target cell populations. The results of the ⁵¹Cr release assays can be used to determine which epitopes of NS3NS4NS5 or E2NS3NS4NS5 are responsible for the strongest CTL response to HCV. NS3*NS4NS5t fusion proteins or E2NS3*NS4NS5t fusion proteins, with or without core polypeptides, which contain the epitopes responsible for the strongest CTL response can then be constructed using the information derived from the ⁵¹Cr release assays.

An HCV fusion protein as described above, or polynucleotide encoding such a fusion protein, can be administered to a mammal, such as a mouse, baboon, chimpanzee, or human, to stimulate a humoral and/or cellular immune response, such as to activate HCV-specific T cells in vivo. Administration can be by any means known in the art, including parenteral, intranasal, intramuscular or subcutaneous injection, including injection using a biological ballistic gun (“gene gun”), as discussed above.

Preferably, injection of an HCV polynucleotide is used to activate T cells. In addition to the practical advantages of simplicity of construction and modification, injection of the polynucleotides results in the synthesis of a fusion protein in the host. Thus, these immunogens are presented to the host immune system with native post-translational modifications, structure, and conformation. The polynucleotides are preferably injected intramuscularly to a large mammal, such as a human, at a dose of 0.5, 0.75, 1.0, 1.5, 2.0, 2.5, 5 or 10 mg/kg.

A composition of the invention comprising an HCV fusion protein or polynucleotide is administered in a manner compatible with the particular composition used and in an amount which is effective to activate HCV-specific T cells as measured by, inter alia, a ⁵¹Cr release assay, a lymphoproliferation assay, or by intracellular staining for IFN-γ. The proteins and/or polynucleotides can be administered either to a mammal which is not infected with an HCV or can be administered to an HCV-infected mammal. The particular dosages of the polynucleotides or fusion proteins in a composition will depend on many factors including, but not limited to the species, age, and general condition of the mammal to which the composition is administered, and the mode of administration of the composition. An effective amount of the composition of the invention can be readily determined using only routine experimentation. In vitro and in vivo models described above can be employed to identify appropriate doses. The amount of polynucleotide used in the example described below provides general guidance which can be used to optimize the activation of HCV-specific T cells either in vivo or in vitro. Generally, 0.5, 0.75, 1.0, 1.5, 2.0, 2.5, 5 or 10 mg of an HCV fusion protein or polynucleotide, with or without a core polypeptide, will be administered to a large mammal, such as a baboon, chimpanzee, or human. If desired, co-stimulatory molecules or adjuvants can also be provided before, after, or together with the compositions.

Immune responses of the mammal generated by the delivery of a composition of the invention, including activation of HCV-specific T cells, can be enhanced by varying the dosage, route of administration, or boosting regimens. Compositions of the invention may be given in a single dose schedule, or preferably in a multiple dose schedule in which a primary course of vaccination includes 1-10 separate doses, followed by other doses given at subsequent time intervals required to maintain and/or reinforce an immune response, for example, at 1-4 months for a second dose, and if needed, a subsequent dose or doses after several months.

### III. EXPERIMENTAL

Below are examples of specific embodiments for carrying out the present invention. The examples are offered for illustrative purposes only, and are not intended to limit the scope of the present invention in any way. Those of skill in the art will readily appreciate that the invention may be practiced in a variety of ways given the teaching of this disclosure.

Efforts have been made to ensure accuracy with respect to numbers used (e.g., amounts, temperatures, etc.), but some experimental error and deviation should, of course, be allowed for.

### Example 1

### Production of NS5Core and NS5tCore Polynucleotides and Polypeptides

NS5t in the following examples represents a C-terminally truncated NS5 molecule, that includes amino acids corresponding to amino acids 1973-2990, numbered relative to the full-length HCV-1 polyprotein.

A polynucleotide encoding NS5t was prepared using standard recombinant techniques and this construct was fused with a polynucleotide encoding a core polypeptide that included amino acids 1-121 of the full-length polyprotein, as depicted at amino acid positions 1772-1892 of FIG. 3, to render NS5tCore121.

The NS5tCore121 polynucleotide was cloned and expressed in S. cerevisiae. In particular, The NS5Core proteins were genetically engineered for expression in S. cerevisiae using the yeast expression vector pBS24.1. This vector contains the 2μsequence for autonomous replication in yeast and the yeast genes leu2d and URA3 as selectable markers. The β-lactamase gene and the ColE1 origin of replication, required for plasmid replication in bacteria, are also present in this expression vector, as well as the α-factor terminator. Expression of the recombinant proteins is under the control of the hybrid ADH2/GAPDH promoter.

Synthetic oligonucleotides (27 bp) with HindIII-EcoNI restriction ends were used at the junction between the ADH2/GAPDH promoter and the HCV-1 NS5a. A 2893 by EcoNI-NdeI restriction fragment encoding NS5a and part of NS5b was gel-purified from pd.Δns3 ns5Pjcore121RT (described in PCT Publication No. WO 01/38360). Synthetic oligonucleotides (205 bp) with NdeI and Nod ends were used for the junction between NS5b-truncated and core. A 318 by NotI-SalI restriction fragment for core121 was gel-purified from pT7Blue2.HCV121 (described in PCT Publication No. WO 01/38360). The entire 3442 by HindIII-SalI polynucleotide encoding NS5tCore121 was subcloned into a pSP72 (Promega, Madison, Wis.) HindIII-SalI vector and sequence-verified. Then, the NS5tCore121 polynucleotide was ligated with the ADH2/GAPDH promoter into the pBS24.1 yeast expression vector.

S. cerevisiae strain AD3 (matα,leu2,trp1,ura3-52,prb-1122,pep4-3,prc1-407,cir^{∘},trp+,:DM15[GAP/ADR]) was transformed with the yeast expression plasmids and single transformants were checked for expression after depletion of glucose in the medium. The cell pellets were lysed with glass beads. Aliquots of the soluble and insoluble fractions were boiled in SDS sample buffer+50 mM DTT, run on 4-20% Tris-Glycine gels, and stained with Coomassie blue. The recombinant proteins were detected in the samples from the insoluble fraction after glass bead lysis.

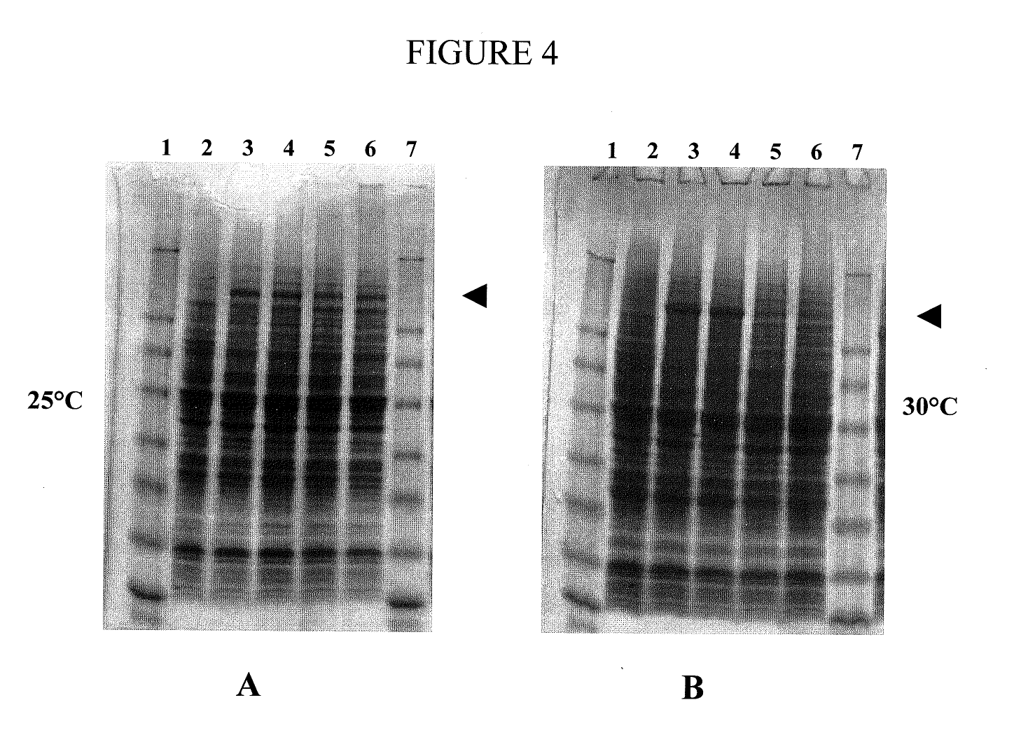

The expression of NS5tCore121 was compared to expression of NS5Core121, a construct including the full-length NS5 sequence (amino acids 1973-3011, numbered relative to the full-length HCV-1 polyprotein) at 25° C. and 30° C. As shown in FIGS. 4A and 4B, expression of the construct including NS5t was greater than expression of the construct including the full-length NS5 sequence.

### Example 2

### Production of NS3*NS4NS5t and NS3*NS4NS5tCore Polynucleotides and Polypeptides

NS3*in the following examples represents a modified NS3 molecule with an alanine substituted for the serine normally found at position 1165, numbered relative to the full-length HCV-1 polyprotein sequence.

A polynucleotide encoding NS3NS4 (approximately amino acids 1027 to 1972, numbered relative to HCV-1) (also termed “NS34” herein) is isolated from an HCV. The NS3 portion of the molecule is mutagenzied by mutating the coding sequence for the Ser residue found at position 1165 to the coding sequence for Ala, such that the resulting molecule lacks NS3 protease activity. This construct is fused with the polynucleotide encoding NS5tCore121 described in Example 1, to render NS3*NS4NS5tCore121. Alternatively, this molecule is fused with NS5t to produce NS3*NS4NS5t. The constructs are cloned into plasmid, vaccinia virus, and adenovirus vectors. Additionally, the constructs are inserted into a recombinant expression vector and used to transform host cells to produce the NS3*NS4NS5tCore121 and NS3*NS4NS5t fusion proteins.

Protease enzyme activity is determined as follows. An NS4A peptide (KKGSVVIVGRIVLSGKPAIIPKK), and the fusion protein of interest are diluted in 90 μl of reaction buffer (25 mM Tris, pH 7.5, 0.15M NaCl, 0.5 mM EDTA, 10% glycerol, 0.05 n-Dodecyl B-D-Maltoside, 5 mM DTT) and allowed to mix for 30 minutes at room temperature. 90 μl of the mixture is added to a microtiter plate (Costar, Inc., Corning, N.Y.) and 10 μl of HCV substrate (AnaSpec, Inc., San Jose Calif.) is added. The plate is mixed and read on a Fluostar plate reader. Results are expressed as relative fluorescence units (RFU) per minute.

### Example 3

### Production of E2NS3*NS4NS5t and E2NS3*NS4NS5tCore Polynucleotides and Polypeptides

E2 in the following examples represents a C-terminally truncated E2 molecule that includes amino acids 384-715, numbered relative to the full-length HCV-1 polyprotein. A polynucleotide encoding the truncated E2 molecule is produced using the methods described in U.S. Pat. Nos. 6,121,020 and 6,326,171, incorporated herein by reference in their entireties. Polynucleotides encoding NS3*NS4NS5tCore121 or NS3*NS4NS5t are produced as described in Example 2. The constructs are fused to render E2NS3*NS4NS5tCore121 and E2NS3*NS4NS5t. The constructs are cloned into plasmid, vaccinia virus, and adenovirus vectors. Additionally, the constructs are inserted into a recombinant expression vector and used to transform host cells to produce the E2NS3*NS4NS5tCore121 and E2NS3*NS4NS5t fusion proteins. Protease enzyme activity is determined as described above.

### Example 4

### Priming of HCV-specific CTLs in Vaccinated Animals

The HCV fusion proteins, NS3*NS4NS5tCore121, NS3*NS4NS5t, E2NS3*NS4NS5tCore121 and E2NS3*NS4NS5t, produced as described above, are used to produce HCV fusion-ISCOMs as follows. The fusion-ISCOM formulations are prepared by mixing the desired fusion protein with a preformed ISCOMATRIX (empty ISCOMs) utilizing ionic interactions to maximize association between the fusion protein and the adjuvant. ISCOMATRIX is prepared essentially as described in Coulter et al. (1998) Vaccine 16:1243.

Rhesus macaques are immunized under anesthesia. Animals are divided into two groups. The first group is infected with 2×10⁸ plaque forming units (pfu) (1×10⁸ intradermally and 1×10⁸ by scarification) of rVVC/E1 at month 0. This group serves as a positive control for CTL priming. Animals from the second group are immunized with 25-100 μg of an HCV fusion polypeptide, as described above, that has been adsorbed to an ISCOM, by intramuscular (IM) injection in the left quadriceps at months 0, 1, 2 and 6. Cytotoxic activity is assayed in a standard ⁵¹Cr release assay as described in, e.g., Paliard et al. (2000) AIDS Res. Hum. Retroviruses 16:273.

### Example 5

### Immunization with the Fusion Polynucleotides

In one immunization protocol, animals are immunized with 50-250 μg of plasmid DNA encoding NS3*NS4NS5tCore121, NS3*NS4NS5t, E2NS3*NS4NS5tCore121 or E2NS3*NS4NS5t by intramuscular injection into the tibialis anterior. A booster injection of 10⁷ pfu of vaccinia virus (VV) encoding NS5a (intraperitoneal), NS3*NS4NS5tCore121, NS3*NS4NS5t, E2NS3*NS4NS5tCore121 or E2NS3*NS4NS5t, or 50-250 μg of plasmid control (intramuscular) is provided 6 weeks later.

In another immunization protocol, animals are injected intramuscularly in the tibialis anterior with 10¹⁰ adenovirus particles encoding NS3*NS4NS5tCore121, NS3*NS4NS5t, E2NS3*NS4NS5tCore121 or E2NS3*NS4NS5t. An intraperitoneal booster injection of 10⁷ pfu of VV-NS5a, or an intramuscular booster injection of 10¹⁰ adenovirus particles encoding NS3*NS4NS5tCore121, NS3*NS4NS5t, E2NS3*NS4NS5tCore121 or E2NS3*NS4NS5t is provided 6 weeks later.

### Example 6

### Activation of HCV-Specific CD8⁺T Cells

⁵¹Cr Release Assay. A ⁵¹Cr release assay is used to measure the ability of HCV-specific T cells to lyse target cells displaying an NS5a epitope. Spleen cells are pooled from the immunized animals. These cells are restimulated in vitro for 6 days with the CTL epitopic peptide p214K9 (2152-HEYPVGSQL-2160; SEQ ID NO:1) from HCV-NS5a in the presence of IL-2. The spleen cells are then assayed for cytotoxic activity in a standard ⁵¹Cr release assay against peptide-sensitized target cells (L929) expressing class I, but not class II MHC molecules, as described in Weiss (1980) J. Biol. Chem. 255:9912-9917. Ratios of effector (T cells) to target (B cells) of 60:1, 20:1, and 7:1 are tested. Percent specific lysis is calculated for each effector to target ratio.

### Example 7

### Activation of HCV-Specific CD8+ T Cells Which Express IFN-γ

Intracellular Staining for Interferon-gamma (IFNγ). Intracellular staining for IFN-γ is used to identify the CD8⁺T cells that secrete IFN-γ after in vitro stimulation with the NS5a epitope p214K9. Spleen cells of individual immunized animals are restimulated in vitro either with p214K9 or with a non-specific peptide for 6-12 hours in the presence of IL-2 and monensin. The cells are then stained for surface CD8 and for intracellular IFN-γ and analyzed by flow cytometry. The percent of CD8⁺T cells which are also positive for IFN-γ is then calculated.

### Example 8

### Proliferation of HCV-Specific CD4⁺T Cells

Lymphoproliferation assay. Spleen cells from pooled immunized animals are depleted of CD8⁺T cells using magnetic beads and are cultured in triplicate with either p222D, an NS5a-epitopic peptide from HCV-NS5a (2224-AELIEANLLWRQEMG-2238; SEQ ID NO:2), or in medium alone. After 72 hours, cells are pulsed with 1μ, Ci per well of ³H-thymidine and harvested 6-8 hours later. Incorporation of radioactivity is measured after harvesting. The mean cpm is calculated.

### Example 9

### Ability of Fusion DNA Vaccine Formulations to Prime CTLs

Animals are immunized with either 10-250 μg of plasmid DNA encoding NS3*NS4NS5tCore121, NS3*NS4NS5t, E2NS3*NS4NS5tCore121 or E2NS3*NS4NS5t as described above, with PLG-linked DNA encoding NS3*NS4NS5tCore121, NS3*NS4NS5t, E2NS3*NS4NS5tCore121 or E2NS3*NS4NS5t (see below), or with DNA encoding NS3*NS4NS5tCore121, NS3*NS4NS5t, E2NS3*NS4NS5tCore121 or E2NS3*NS4NS5t, delivered via electroporation (see, e.g., International Publication No. WO/0045823 for this delivery technique). The immunizations are followed by a booster injection 6 weeks later of plasmid DNA encoding NS3*NS4NS5tCore121, NS3*NS4NS5t, E2NS3*NS4NS5tCore121 or E2NS3*NS4NS5t.

PLG-delivered DNA. The polylactide-co-glycolide (PLG) polymers are obtained from Boehringer Ingelheim, U.S.A. The PLG polymer is RG505, which has a copolymer ratio of 50/50 and a molecular weight of 65 kDa (manufacturers data). Cationic microparticles with adsorbed DNA are prepared using a modified solvent evaporation process, essentially as described in Singh et al., Proc. Natl. Acad. Sci. USA (2000) 97:811-816. Briefly, the microparticles are prepared by emulsifying 10 ml of a 5% w/v polymer solution in methylene chloride with 1 ml of PBS at high speed using an IKA homogenizer. The primary emulsion is then added to 50 ml of distilled water containing cetyl trimethyl ammonium bromide (CTAB) (0.5% w/v). This results in the formation of a w/o/w emulsion which is stirred at 6000 rpm for 12 hours at room temperature, allowing the methylene chloride to evaporate. The resulting microparticles are washed twice in distilled water by centrifugation at 10,000 g and freeze dried. Following preparation, washing and collection, DNA constructs are adsorbed onto the microparticles by incubating 100 mg of cationic microparticles in a 1 mg/ml solution of DNA at 4° C. for 6 hours. The microparticles are then separated by centrifugation, the pellet washed with TE buffer and the microparticles are freeze dried.

CTL activity and IFN-γ expression is measured by ⁵¹Cr release assay or intracellular staining as described in the examples above.

### Example 10

### Immunization Routes and Replicon Particles SINCR (DC+) Encoding for the Fusion Proteins

Alphavirus replicon particles, for example, SINCR (DC+) are prepared as described in Polo et al., Proc. Natl. Acad. Sci. USA (1999) 96:4598-4603. Animals are injected with 5×10⁶ IU SINCR (DC+) replicon particles encoding for NS3*45tCore intramuscularly (IM) as described above, or subcutaneously (S/C) at the base of the tail (BoT) and foot pad (FP), or with a combination of ⅔ of the DNA delivered via IM administration and ⅓ via a BoT route. The immunizations are followed by a booster injection of vaccinia virus as described above. IFN-γ expression is measured by intracellular staining as described in the examples above.

### Example 11

### Alphavirus Replicon Priming, Followed by Various Boosting Regimes

Alphavirus replicon particles, for example, SINCR (DC+) are prepared as described in Polo et al., Proc. Natl. Acad. Sci. USA (1999) 96:4598-4603. Animals are primed with SINCR (DC+), 1.5×10⁶ IU replicon particles encoding a fusion protein as described above, by intramuscular injection into the tibialis anterior, followed by a booster of either 10-100 μg of plasmid DNA encoding for NS5a, NS3*NS4NS5tCore121, NS3*NS4NS5t, E2NS3*NS4NS5tCore121 or E2NS3*NS4NS5t, 10¹⁰ adenovirus particles encoding NS3*NS4NS5tCore121, NS3*NS4NS5t, E2NS3*NS4NS5tCore121 or E2NS3*NS4NS5t, 1.5×10⁶ IU SINCR (DC+) replicon particles encoding NS3*NS4NS5tCore121, NS3*NS4NS5t, E2NS3*NS4NS5tCore121 or E2NS3*NS4NS5t, or 10⁷ pfu vaccinia virus encoding NS3*NS4NS5tCore121, NS3*NS4NS5t, E2NS3*NS4NS5tCore121 or E2NS3*NS4NS5t at 6 weeks. IFN-γ expression is measured by intracellular staining as described above.

### Example 12

Alphaviruses Expressing NS3*NS4NS5tCore121, NS3*NS4NS5t, E2NS3*NS4NS5tCore121 or E2NS3*NS4NS5t Alphavirus replicon particles, for example, SINCR (DC+) and SINCR (LP) are prepared as described in Polo et al., Proc. Natl. Acad. Sci. USA (1999) 96:4598-4603. Animals are immunized with 1×10² to 1×10⁶ IU SINCR (DC+) replicons encoding NS3*NS4NS5tCore121, NS3*NS4NS5t, E2NS3*NS4NS5tCore121 or E2NS3*NS4NS5t via a combination of delivery routes (⅔ IM and ⅓ S/C) as well as by S/C alone, or with 1×10² to 1×10⁶ IU SINCR (LP) replicon particles encoding NS3*NS4NS5tCore121, NS3*NS4NS5t, E2NS3*NS4NS5tCore121 or E2NS3*NS4NS5t via a combination of delivery routes (⅔ IM and ⅓ S/C) as well as by S/C alone. The immunizations are followed by a booster injection of 10⁷ pfu vaccinia virus encoding NS5a, NS3*NS4NS5tCore121, NS3*NS4NS5t, E2NS3*NS4NS5tCore121 or E2NS3*NS4NS5t at 6 weeks. IFN-γ expression is measured by intracellular staining as described in Example 5.

Thus, C-terminally truncated HCV NS5 and fusion polypeptides comprising the same, are disclosed. Although preferred embodiments of the subject invention have been described in some detail, it is understood that obvious variations can be made without departing from the spirit and the scope of the invention as defined by the claims.

1. A C-terminally truncated NS5 polypeptide, wherein said polypeptide comprises a full-length NS5a polypeptide and an N-terminal portion of an NS5b polypeptide.

2. The C-terminally truncated NS5 polypeptide of claim 1, wherein the polypeptide is truncated at a position between amino acid 2500 and the C-terminus, numbered relative to the full-length HCV-1 polyprotein.

3. The C-terminally truncated NS5 polypeptide of claim 1, wherein the polypeptide is truncated at a position between amino acid 2900 and the C-terminus, numbered relative to the full-length HCV-1 polyprotein.

4. The C-terminally truncated NS5 polypeptide of claim 3, wherein the polypeptide is truncated at the amino acid corresponding to the amino acid immediately following amino acid 2990, numbered relative to the full-length HCV-1 polyprotein.

5. The C-terminally truncated NS5 polypeptide of claim 4, wherein the polypeptide consists of an amino acid sequence corresponding to amino acids 1973-2990, numbered relative to the full-length HCV-1 polyprotein.

6. An immunogenic fusion protein comprising the C-terminally truncated NS5 polypeptide of claim 1, and at least one polypeptide derived from a region of the HCV polyprotein other than the NS5 region.

7. The fusion protein of claim 6, wherein the protein further comprises a modified NS3 polypeptide comprising a substitution of an amino acid corresponding to His-1083, Asp-1105 and/or Ser-1165, numbered relative to the full-length HCV-1 polyprotein such that protease activity is inhibited when the modified NS3 polypeptide is present in an HCV fusion protein.

8. The fusion protein of claim 7, wherein the modified NS3 polypeptide comprises a substitution of an alanine for the amino acid corresponding to Ser-1165, numbered relative to the full-length HCV-1 polyprotein.

9. The fusion protein of claim 6, wherein the protein comprises a modified NS3 polypeptide, an NS4 polypeptide, and optionally an HCV core polypeptide.

10. The fusion protein of claim 9, wherein the core polypeptide comprises a C-terminal truncation.

11. The fusion protein of claim 10, wherein the core polypeptide consists of the sequence of amino acids depicted at amino acid positions 1772-1892 of SEQ ID NO:6.

12. The fusion protein of claim 6, further comprising an E2 polypeptide.

13. The fusion protein of claim 12, wherein the E2 polypeptide is a C-terminally truncated E2 polypeptide consisting of an amino acid sequence corresponding to amino acids 384-715, numbered relative to the full-length HCV-1 polyprotein.

14. The fusion protein of claim 6, wherein each of the polypeptides present in the fusion is derived from the same HCV isolate.

15. The fusion protein of claim 6, wherein at least one of the polypeptides present in the fusion is derived from a different isolate than the C-terminally truncated NS5 polypeptide.

16. An immunogenic fusion protein consisting essentially of, in amino terminal to carboxy terminal direction:
(a) a modified NS3 polypeptide comprising a substitution of an alanine for the amino acid corresponding to Ser-1165, numbered relative to the full-length HCV-1 polyprotein such that protease activity is inhibited;
(b) an NS4 polypeptide;
(c) a C-terminally truncated NS5 polypeptide, wherein the NS5 polypeptide consists of an amino acid sequence corresponding to amino acids 1973-2990, numbered relative to the full-length HCV-1 polyprotein; and
(d) optionally, an HCV core polypeptide.

17. The fusion protein of claim 16, wherein the fusion protein comprises an HCV core polypeptide.

18. The fusion protein of claim 17, wherein the core polypeptide comprises a C-terminal truncation.

19. The fusion protein of claim 18, wherein the core polypeptide consists of the sequence of amino acids depicted at amino acid positions 1772-1892 of SEQ ID NO:6.

20. An immunogenic fusion protein consisting essentially of, in amino terminal to carboxy terminal direction:
(a) a C-terminally truncated E2 polypeptide consisting of an amino acid sequence corresponding to amino acids 384-715, numbered relative to the full-length HCV-1 polyprotein;
(b) a modified NS3 polypeptide comprising a substitution of an alanine for the amino acid corresponding to Ser-1165, numbered relative to the full-length HCV-1 polyprotein such that protease activity is inhibited;
(c) an NS4 polypeptide;
(d) a C-terminally truncated NS5 polypeptide, wherein the NS5 polypeptide consists of an amino acid sequence corresponding to amino acids 1973-2990, numbered relative to the full-length HCV-1 polyprotein; and
(e) optionally, an HCV core polypeptide.

21. The fusion protein of claim 20, wherein the fusion protein comprises an HCV core polypeptide.

22. The fusion protein of claim 21, wherein the core polypeptide comprises a C-terminal truncation.

23. The fusion protein of claim 22, wherein the core polypeptide consists of the sequence of amino acids depicted at amino acid positions 1772-1892 of SEQ ID NO:6.

24. A composition comprising a C-terminally truncated NS5 polypeptide according to claim 1 in combination with a pharmaceutically acceptable excipient.

25. A composition comprising an immunogenic fusion protein according to claim 6 in combination with a pharmaceutically acceptable excipient.

26. A method of stimulating a cellular immune response in a vertebrate subject comprising administering to the subject a therapeutically effective amount of the composition of claim 24.

27. A method of stimulating a cellular immune response in a vertebrate subject comprising administering to the subject a therapeutically effective amount of the composition of claim 25.

28. A polynucleotide comprising a coding sequence encoding a C-terminally truncated NS5 polypeptide according to claim 1.

29. A polynucleotide comprising a coding sequence encoding an immunogenic fusion protein according to claim 6.

30. A recombinant vector comprising:
(a) a polynucleotide according to claim 28; and
(b) at least one control element operably linked to said polynucleotide, whereby said coding sequence can be transcribed and translated in a host cell.

31. A recombinant vector comprising:
(a) a polynucleotide according to claim 29; and
(b) at least one control element operably linked to said polynucleotide, whereby said coding sequence can be transcribed and translated in a host cell.

32. A host cell comprising the recombinant vector of claim 30.

33. A host cell comprising the recombinant vector of claim 31.

34. A method for producing an immunogenic C-terminally truncated NS5 polypeptide or an immunogenic fusion protein comprising said polypeptide, said method comprising culturing a population of host cells according to claim 32 under conditions for producing said protein.

35. A method for producing an immunogenic C-terminally truncated NS5 polypeptide or an immunogenic fusion protein comprising said polypeptide, said method comprising culturing a population of host cells according to claim 33 under conditions for producing said protein.

36. A method for enhancing production of an HCV NS5 polypeptide comprising culturing a population of host cells according to claim 32 under conditions for producing said protein, wherein said protein is produced in greater amounts as compared to the amount of a full-length NS5 polypeptide produced under the same conditions.


SEQUENCE LISTING (9 sequences)

SEQ ID NO: 1 - protein, 9 aa, Artificial Sequence
  synthetic epitope recognized by a Tcell receptor
HEYPVGSQL

SEQ ID NO: 2 - protein, 15 aa, Artificial Sequence
  synthetic epitope recognized by a Tcell receptor
AELIEANLLWRQEMG

SEQ ID NO: 3 - DNA, 546 bp, Hepatitis C virus
  CDS (1)..(546)
atggcgcccatcacggcgtacgcccagcagacaaggggcctcctagggtgcataatcaccagcctaactggccgggacaaaaaccaagtggagggtgaggtccagattgtgtcaactgctgcccaaaccttcctggcaacgtgcatcaatggggtgtgctggactgtctaccacggggccggaacgaggaccatcgcgtcacccaagggtcctgtcatccagatgtataccaatgtagaccaagaccttgtgggctggcccgctccgcaaggtagccgatcattgacaccctgcacttgcggctcctcggacctttacctggtcacgaggcacgccgatgtcattcccgtgcgccggcggggtgatagcaggggcagcctgctgtcgccccggcccatttcctacttgaaaggctcctcggggggtccgctgttgtgccccgcggggcacgccgtgggcatatttagggccgcggtgtgcacccgtggagtggctaaggcggtggactttatccctgtggagaacctagagacaaccatgaggtcc

SEQ ID NO: 4 - protein, 182 aa, Hepatitis C virus
MAPITAYAQQTRGLLGCIITSLTGRDKNQVEGEVQIVSTAAQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVENLETTMRS

SEQ ID NO: 5 - DNA, 5676 bp, Artificial Sequence
  synthetic DNA sequence of a representative modified fusion protein, with the NS3 protease domain deleted from the N-terminus and including amino acids 1-121 of Core on the C-terminus
atggctgcatatgcagctcagggctataaggtgctagtactcaacccctctgttgctgcaacactgggctttggtgcttacatgtccaaggctcatgggatcgatcctaacatcaggaccggggtgagaacaattaccactggcagccccatcacgtactccacctacggcaagttccttgccgacggcgggtgctcggggggcgcttatgacataataatttgtgacgagtgccactccacggatgccacatccatcttgggcattggcactgtccttgaccaagcagagactgcgggggcgagactggttgtgctcgccaccgccacccctccgggctccgtcactgtgccccatcccaacatcgaggaggttgctctgtccaccaccggagagatccctttttacggcaaggctatccccctcgaagtaatcaagggggggagacatctcatcttctgtcattcaaagaagaagtgcgacgaactcgccgcaaagctggtcgcattgggcatcaatgccgtggcctactaccgcggtcttgacgtgtccgtcatcccgaccagcggcgatgttgtcgtcgtggcaaccgatgccctcatgaccggctataccggcgacttcgactcggtgatagactgcaatacgtgtgtcacccagacagtcgatttcagccttgaccctaccttcaccattgagacaatcacgctcccccaagatgctgtctcccgcactcaacgtcggggcaggactggcagggggaagccaggcatctacagatttgtggcaccgggggagcgcccctccggcatgttcgactcgtccgtcctctgtgagtgctatgacgcaggctgtgcttggtatgagctcacgcccgccgagactacagttaggctacgagcgtacatgaacaccccggggcttcccgtgtgccaggaccatcttgaattttgggagggcgtctttacaggcctcactcatatagatgcccactttctatcccagacaaagcagagtggggagaaccttccttacctggtagcgtaccaagccaccgtgtgcgctagggctcaagcccctcccccatcgtgggaccagatgtggaagtgtttgattcgcctcaagcccaccctccatgggccaacacccctgctatacagactgggcgctgttcagaatgaaatcaccctgacgcacccagtcaccaaatacatcatgacatgcatgtcggccgacctggaggtcgtcacgagcacctgggtgctcgttggcggcgtcctggctgctttggccgcgtattgcctgtcaacaggctgcgtggtcatagtgggcagggtcgtcttgtccgggaagccggcaatcatacctgacagggaagtcctctaccgagagttcgatgagatggaagagtgctctcagcacttaccgtacatcgagcaagggatgatgctcgccgagcagttcaagcagaaggccctcggcctcctgcagaccgcgtcccgtcaggcagaggttatcgcccctgctgtccagaccaactggcaaaaactcgagaccttctgggcgaagcatatgtggaacttcatcagtgggatacaatacttggcgggcttgtcaacgctgcctggtaaccccgccattgcttcattgatggcttttacagctgctgtcaccagcccactaaccactagccaaaccctcctcttcaacatattgggggggtgggtggctgcccagctcgccgcccccggtgccgctactgcctttgtgggcgctggcttagctggcgccgccatcggcagtgttggactggggaaggtcctcatagacatccttgcagggtatggcgcgggcgtggcgggagctcttgtggcattcaagatcatgagcggtgaggtcccctccacggaggacctggtcaatctactgcccgccatcctctcgcccggagccctcgtagtcggcgtggtctgtgcagcaatactgcgccggcacgttggcccgggcgagggggcagtgcagtggatgaaccggctgatagccttcgcctcccgggggaaccatgtttcccccacgcactacgtgccggagagcgatgcagctgcccgcgtcactgccatactcagcagcctcactgtaacccagctcctgaggcgactgcaccagtggataagctcggagtgtaccactccatgctccggttcctggctaagggacatctgggactggatatgcgaggtgttgagcgactttaagacctggctaaaagctaagctcatgccacagctgcctgggatcccctttgtgtcctgccagcgcgggtataagggggtctggcgaggggacggcatcatgcacactcgctgccactgtggagctgagatcactggacatgtcaaaaacgggacgatgaggatcgtcggtcctaggacctgcaggaacatgtggagtgggaccttccccattaatgcctacaccacgggcccctgtaccccccttcctgcgccgaactacacgttcgcgctatggagggtgtctgcagaggaatacgtggagataaggcaggtgggggacttccactacgtgacgggtatgactactgacaatcttaaatgcccgtgccaggtcccatcgcccgaatttttcacagaattggacggggtgcgcctacataggtttgcgcccccctgcaagcccttgctgcgggaggaggtatcattcagagtaggactccacgaatacccggtagggtcgcaattaccttgcgagcccgaaccggacgtggccgtgttgacgtccatgctcactgatccctcccatataacagcagaggcggccgggcgaaggttggcgaggggatcacccccctctgtggccagctcctcggctagccagctatccgctccatctctcaaggcaacttgcaccgctaaccatgactcccctgatgctgagctcatagaggccaacctcctatggaggcaggagatgggcggcaacatcaccagggttgagtcagaaaacaaagtggtgattctggactccttcgatccgcttgtggcggaggaggacgagcgggagatctccgtacccgcagaaatcctgcggaagtctcggagattcgcccaggccctgcccgtttgggcgcggccggactataaccccccgctagtggagacgtggaaaaagcccgactacgaaccacctgtggtccatggctgcccgcttccacctccaaagtcccctcctgtgcctccgcctcggaagaagcggacggtggtcctcactgaatcaaccctatctactgccttggccgagctcgccaccagaagctttggcagctcctcaacttccggcattacgggcgacaatacgacaacatcctctgagcccgccccttctggctgcccccccgactccgacgctgagtcctattcctccatgccccccctggagggggagcctggggatccggatcttagcgacgggtcatggtcaacggtcagtagtgaggccaacgcggaggatgtcgtgtgctgctcaatgtcttactcttggacaggcgcactcgtcaccccgtgcgccgcggaagaacagaaactgcccatcaatgcactaagcaactcgttgctacgtcaccacaatttggtgtattccaccacctcacgcagtgcttgccaaaggcagaagaaagtcacatttgacagactgcaagttctggacagccattaccaggacgtactcaaggaggttaaagcagcggcgtcaaaagtgaaggctaacttgctatccgtagaggaagcttgcagcctgacgcccccacactcagccaaatccaagtttggttatggggcaaaagacgtccgttgccatgccagaaaggccgtaacccacatcaactccgtgtggaaagaccttctggaagacaatgtaacaccaatagacactaccatcatggctaagaacgaggttttctgcgttcagcctgagaaggggggtcgtaagccagctcgtctcatcgtgttccccgatctgggcgtgcgcgtgtgcgaaaagatggctttgtacgacgtggttacaaagctccccttggccgtgatgggaagctcctacggattccaatactcaccaggacagcgggttgaattcctcgtgcaagcgtggaagtccaagaaaaccccaatggggttctcgtatgatacccgctgctttgactccacagtcactgagagcgacatccgtacggaggaggcaatctaccaatgttgtgacctcgacccccaagcccgcgtggccatcaagtccctcaccgagaggctttatgttgggggccctcttaccaattcaaggggggagaactgcggctatcgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagcgcgggggtccaggaggacgcggcgagcctgagagccttcacggaggctatgaccaggtactccgccccccctggggaccccccacaaccagaatacgacttggagctcataacatcatgctcctccaacgtgtcagtcgcccacgacggcgctggaaagagggtctactacctcacccgtgaccctacaacccccctcgcgagagctgcgtgggagacagcaagacacactccagtcaattcctggctaggcaacataatcatgtttgcccccacactgtgggcgaggatgatactgatgacccatttctttagcgtccttatagccagggaccagcttgaacaggccctcgattgcgagatctacggggcctgctactccatagaaccactggatctacctccaatcattcaaagactccatggcctcagcgcattttcactccacagttactctccaggtgaaatcaatagggtggccgcatgcctcagaaaacttggggtaccgcccttgcgagcttggagacaccgggcccggagcgtccgcgctaggcttctggccagaggaggcagggctgccatatgtggcaagtacctcttcaactgggcagtaagaacaaagctcaaactcactccaatagcggccgctggccagctggacttgtccggctggttcacggctggctacagcgggggagacatttatcacagcgtgtctcatgcccggccccgctggatctggttttgcctactcctgcttgctgcaggggtaggcatctacctcctccccaaccgaatgagcacgaatcctaaacctcaaagaaagaccaaacgtaacaccaaccggcggccgcaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaag

SEQ ID NO: 6 - protein, 1892 aa, Artificial Sequence
  synthetic amino acid sequence of a representative modified fusion protein, with the NS3 protease domain deleted from the N-terminus and including amino acids 1-121 of Core on the C-terminus
MAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVTSTWVLVGGVLAALAAYCLSTGCVVIVGRVVLSGKPAIIPDREVLYREFDEMEECSQHLPYIEQGMMLAEQFKQKALGLLQTASRQAEVIAPAVQTNWQKLETFWAKHMWNFISGIQYLAGLSTLPGNPAIASLMAFTAAVTSPLTTSQTLLFNILGGWVAAQLAAPGAATAFVGAGLAGAAIGSVGLGKVLIDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYKGVWRGDGIMHTRCHCGAEITGHVKNGTMRIVGPRTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSVASSSASQLSAPSLKATCTANHDSPDAELIEANLLWRQEMGGNITRVESENKVVILDSFDPLVAEEDEREISVPAEILRKSRRFAQALPVWARPDYNPPLVETWKKPDYEPPVVHGCPLPPPKSPPVPPPRKKRTVVLTESTLSTALAELATRSFGSSSTSGITGDNTTTSSEPAPSGCPPDSDAESYSSMPPLEGEPGDPDLSDGSWSTVSSEANAEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRWIWFCLLLLAAGVGIYLLPNRMSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGK

SEQ ID NO: 7 - DNA, 3417 bp, Artificial Sequence
  synthetic DNA sequence of a representative fusion protein that includes a C-terminally truncated NS5 polypeptide with the C-terminus of the NS5 polypeptide fused to a core polypeptide
tccggttcctggctaagggacatctgggactggatatgcgaggtgttgagcgactttaagacctggctaaaagctaagctcatgccacagctgcctgggatcccctttgtgtcctgccagcgcgggtataagggggtctggcgaggggacggcatcatgcacactcgctgccactgtggagctgagatcactggacatgtcaaaaacgggacgatgaggatcgtcggtcctaggacctgcaggaacatgtggagtgggaccttccccattaatgcctacaccacgggcccctgtaccccccttcctgcgccgaactacacgttcgcgctatggagggtgtctgcagaggaatacgtggagataaggcaggtgggggacttccactacgtgacgggtatgactactgacaatcttaaatgcccgtgccaggtcccatcgcccgaatttttcacagaattggacggggtgcgcctacataggtttgcgcccccctgcaagcccttgctgcgggaggaggtatcattcagagtaggactccacgaatacccggtagggtcgcaattaccttgcgagcccgaaccggacgtggccgtgttgacgtccatgctcactgatccctcccatataacagcagaggcggccgggcgaaggttggcgaggggatcacccccctctgtggccagctcctcggctagccagctatccgctccatctctcaaggcaacttgcaccgctaaccatgactcccctgatgctgagctcatagaggccaacctcctatggaggcaggagatgggcggcaacatcaccagggttgagtcagaaaacaaagtggtgattctggactccttcgatccgcttgtggcggaggaggacgagcgggagatctccgtacccgcagaaatcctgcggaagtctcggagattcgcccaggccctgcccgtttgggcgcggccggactataaccccccgctagtggagacgtggaaaaagcccgactacgaaccacctgtggtccatggctgcccgcttccacctccaaagtcccctcctgtgcctccgcctcggaagaagcggacggtggtcctcactgaatcaaccctatctactgccttggccgagctcgccaccagaagctttggcagctcctcaacttccggcattacgggcgacaatacgacaacatcctctgagcccgccccttctggctgcccccccgactccgacgctgagtcctattcctccatgccccccctggagggggagcctggggatccggatcttagcgacgggtcatggtcaacggtcagtagtgaggccaacgcggaggatgtcgtgtgctgctcaatgtcttactcttggacaggcgcactcgtcaccccgtgcgccgcggaagaacagaaactgcccatcaatgcactaagcaactcgttgctacgtcaccacaatttggtgtattccaccacctcacgcagtgcttgccaaaggcagaagaaagtcacatttgacagactgcaagttctggacagccattaccaggacgtactcaaggaggttaaagcagcggcgtcaaaagtgaaggctaacttgctatccgtagaggaagcttgcagcctgacgcccccacactcagccaaatccaagtttggttatggggcaaaagacgtccgttgccatgccagaaaggccgtaacccacatcaactccgtgtggaaagaccttctggaagacaatgtaacaccaatagacactaccatcatggctaagaacgaggttttctgcgttcagcctgagaaggggggtcgtaagccagctcgtctcatcgtgttccccgatctgggcgtgcgcgtgtgcgaaaagatggctttgtacgacgtggttacaaagctccccttggccgtgatgggaagctcctacggattccaatactcaccaggacagcgggttgaattcctcgtgcaagcgtggaagtccaagaaaaccccaatggggttctcgtatgatacccgctgctttgactccacagtcactgagagcgacatccgtacggaggaggcaatctaccaatgttgtgacctcgacccccaagcccgcgtggccatcaagtccctcaccgagaggctttatgttgggggccctcttaccaattcaaggggggagaactgcggctatcgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagcgcgggggtccaggaggacgcggcgagcctgagagccttcacggaggctatgaccaggtactccgccccccctggggaccccccacaaccagaatacgacttggagctcataacatcatgctcctccaacgtgtcagtcgcccacgacggcgctggaaagagggtctactacctcacccgtgaccctacaacccccctcgcgagagctgcgtgggagacagcaagacacactccagtcaattcctggctaggcaacataatcatgtttgcccccacactgtgggcgaggatgatactgatgacccatttctttagcgtccttatagccagggaccagcttgaacaggccctcgattgcgagatctacggggcctgctactccatagaaccactggatctacctccaatcattcaaagactccatggcctcagcgcattttcactccacagttactctccaggtgaaatcaatagggtggccgcatgcctcagaaaacttggggtaccgcccttgcgagcttggagacaccgggcccggagcgtccgcgctaggcttctggccagaggaggcagggctgccatatgtggcaagtacctcttcaactgggcagtaagaacaaagctcaaactcactccaatagcggccgctggccagctggacttgtccggctggttcacggctggctacagcgggggagacatttatcacagcgtgtctcatgcccggccccgcatgagcacgaatcctaaacctcaaagaaagaccaaacgtaacaccaaccggcggccgcaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaag

SEQ ID NO: 8 - protein, 1139 aa, Artificial Sequence
  synthetic amino acid sequence of a representative fusion protein that includes a C-terminally truncated NS5 polypeptide with the C-terminus of the NS5 polypeptide fused to a core polypeptide
SGSWLRDIWDWICEVLSDFKTWLKAKLMPQLPGIPFVSCQRGYKGVWRGDGIMHTRCHCGAEITGHVKNGTMRIVGPRTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSVASSSASQLSAPSLKATCTANHDSPDAELIEANLLWRQEMGGNITRVESENKVVILDSFDPLVAEEDEREISVPAEILRKSRRFAQALPVWARPDYNPPLVETWKKPDYEPPVVHGCPLPPPKSPPVPPPRKKRTVVLTESTLSTALAELATRSFGSSSTSGITGDNTTTSSEPAPSGCPPDSDAESYSSMPPLEGEPGDPDLSDGSWSTVSSEANAEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRMSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGK

SEQ ID NO: 9 - protein, 3011 aa, Hepatitis C virus
  misc_feature: Hepatitis C Virus genotype 1a
MSTNPKPQKKNKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNASRCWVAMTPTVATRDGKLPATQLRRHIDLLVGSATLCSALYVGDLCGSVFLVGQLFTFSPRRHWTTQGCNCSIYPGHITGHRMAWDMMMNWSPTTALVMAQLLRIPQAILDMIAGAHWGVLAGIAYFSMVGNWAKVLVVLLLFAGVDAETHVTGGSAGHTVSGFVSLLAPGAKQNVQLINTNGSWHLNSTALNCNDSLNTGWLAGLFYHHKFNSSGCPERLASCRPLTDFDQGWGPISYANGSGPDQRPYCWHYPPKPCGIVPAKSVCGPVYCFTPSPVVVGTTDRSGAPTYSWGENDTDVFVLNNTRPPLGNWFGCTWMNSTGFTKVCGAPPCVIGGAGNNTLHCPTDCFRKHPDATYSRCGSGPWITPRCLVDYPYRLWHYPCTINYTIFKIRMYVGGVEHRLEAACNWTRGERCDLEDRDRSELSPLLLTTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGKWVPGAVYTFYGMWPLLLLLLALPQRAYALDTEVAASCGGVVLVGLMALTLSPYYKRYISWCLWWLQYFLTRVEAQLHVWIPPLNVRGGRDAVILLMCAVHPTLVFDITKLLLAVFGPLWILQASLLKVPYFVRVQGLLRFCALARKMIGGHYVQMVIIKLGALTGTYVYNHLTPLRDWAHNGLRDLAVAVEPVVFSQMETKLITWGADTAACGDIINGLPVSARRGREILLGPADGMVSKGWRLLAPITAYAQQTRGLLGCIITSLTGRDKNQVEGEVQIVSTAAQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVTSTWVLVGGVLAALAAYCLSTGCVVIVGRVVLSGKPAIIPDREVLYREFDEMEECSQHLPYIEQGMMLAEQFKQKALGLLQTASRQAEVIAPAVQTNWQKLETFWAKHMWNFISGIQYLAGLSTLPGNPAIASLMAFTAAVTSPLTTSQTLLFNILGGWVAAQLAAPGAATAFVGAGLAGAAIGSVGLGKVLIDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYKGVWRVDGIMHTRCHCGAEITGHVKNGTMRIVGPRTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSVASSSASQLSAPSLKATCTANHDSPDAELIEANLLWRQEMGGNITRVESENKVVILDSFDPLVAEEDEREISVPAEILRKSRRFAQALPVWARPDYNPPLVETWKKPDYEPPVVHGCPLPPPKSPPVPPPRKKRTVVLTESTLSTALAELATRSFGSSSTSGITGDNTTTSSEPAPSGCPPDSDAESYSSMPPLEGEPGDPDLSDGSWSTVSSEANAEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRWIWFCLLLLAAGVGIYLLPNR